(19) United States
(12) Patent Application Publication
(10) Pub. No.: US 2012/0071346 A1
(43) Pub. Date: Mar. 22, 2012

(54) GENE-BASED ALGORITHMIC CANCER PROGNOSIS

(75) Inventors: Christos SOTIRIOU, Bruxelles (BE); Mauro Delorenzi, Epalinges (CH); Martine Piccart, Brussels (BE); Virginie Durbecq, Carnieres (BE)

(73) Assignee: Universite Libre de Bruxelles, Bruxelles (BE)

(21) Appl. No.: 13/306,590

(22) Filed: Nov. 29, 2011

Related U.S. Application Data
(62) Division of application No. 11/929,043, filed on Oct. 30, 2007.
Application No. 11/929,043 is a continuation-in-part of application No. PCT/BE2006/000051, filed on May 15, 2006.
(60) Provisional application No. 60/680,543, filed on May 13, 2005.

(30) Foreign Application Priority Data
Dec. 7, 2005 (EP) 05447274.1

(51) Int. Cl. C40B 30/04; C40B 60/12; C12M 1/40; C12Q 1/68; C40B 40/06

(52) U.S. Cl. 506/9; 435/6.12; 506/16; 435/287.2; 506/39

(57) ABSTRACT

The present invention is related to the methods and systems for prognosis determination in tumor samples, by measuring gene expression in a tumor sample and applying a gene-expression grade index (GGI) or a relapse score (RS) to yield a number c risk score.

This application is a Divisional Application of U.S. Ser. No. 11/929,043, filed on 30 Oct. 2007, which is a Continuation-in-Part of PCT/BE2006/00051, filed 15 May 2006, which claims benefit of Serial No. 05447274.1, filed 7 Dec. 2005 in the EPO, and which also claims benefit of U.S. Ser. No. 60/680,543, filed 13 May 2005 and which applications are incorporated herein by reference. A claim of priority to all, to the extent appropriate is made.

### FIELD OF THE INVENTION

The present invention is related to new method and tools for improving cancer prognosis.

### BACKGROUND OF THE INVENTION

Micro-array profiling, or the assessment of the mRNA expression levels of hundreds and thousands of genes, has shown that cancer can be divided into distinct molecular subgroups by the expression levels of certain genes. These subgroups seem to have distinct clinical outcomes and also may respond differently to different therapeutic agents used in cancer treatment. But the current understanding of the underlying biology does not permit “individualization” of a particular cancer patients' care. As a result for breast cancer, for example, many women today are given systemic treatments such as chemotherapy or endocrine therapy in an attempt to reduce her risk of the breast cancer recurring after initial diagnosis. Unfortunately, this systemic treatment only benefits a minority of women who will relapse, hence exposing many women to unnecessary and potentially toxic treatment. New prognostic tools developed using micro-array technology show potential in allowing us to facilitate tailored treatment of breast cancer patients (Paik et al, New England Journal of Medicine 351:27 (2004); Van de Vijver et al, New England Journal of Medicine 347:199 (2002); Wang et al, Lancet 365: 671 (2005)). These genomic tools may be a much needed improvement over currently used clinical methods.

Histological grading of breast carcinomas has long been recognised to provide significant clinical prognostic information. However, despite recommendations by the College of American Pathologists for use of tumor grade as a prognostic factor in breast cancer, the latest Breast Task Force serving the American Joint Committee on Cancer (AJCC) did not include it in its staging criteria, citing insurmountable inconsistencies between institutions and lack of data. This may be in part related to inter-observer variability and the various grading approaches used, resulting in poor reproducibility across institutions. With the advent of standardized methods such as those developed by Elston and Ellis, concordance between institutions has been improved. Nevertheless, whilst grade 1 (low risk) and (high risk) are clearly associated with different prognoses, tumors classified as intermediate grade present a difficulty in clinical decision making for treatment because their survival profile is not different from that of the total (non-graded) population and their proportion is large (40%-50%). A more accurate grading system would allow for better prognostication and improved selection of women for further breast cancer treatment.

The majority of breast cancers diagnosed today are hormone responsive. Tamoxifen is the most common anti-estrogen agent prescribed today in the adjuvant treatment of these patients. Yet up to 40% of these patients will relapse when given tamoxifen in this setting. At present, due to the positive results of several large trials evaluating the use of aromatase inhibitors instead of, or in combination or sequence with tamoxifen in the adjuvant setting, there are many options available for post menopausal women with hormone responsive breast cancer. Furthermore, it is unclear which treatment option is the best especially given that the long term health costs of aromatase inhibitor use are unknown. The ability to identify a group at high risk of relapse when given tamoxifen could aid in identifying patients for whom tamoxifen is probably not the best option. These patients could then be specifically targeted for alternative treatment strategies.

Particularly relating to the issue of predicting relapse for women treated with adjuvant tamoxifen, two publications have been reported claiming gene sets that can predict clinical outcome (Ma et al, Cancer Cell 5:607 (2004), Jansen et al. Journal of Clinical Oncology 23:732 (2005). These studies involved small numbers of patients and hence are not thoroughly validated to be widely used clinically.

Accordingly need exists for methods and systems that can accurately assess prognosis and hence help oncologists tailor their treatment decisions for the individual cancer patient. In particular, a need exists for methods and systems directed to breast cancer patients.

### AIMS OF THE INVENTION

The present invention aims to provide new methods and tools for improving cancer prognosis that do not present the drawbacks of the methods of the state of the art.

### SUMMARY OF THE INVENTION

The present invention is related to a gene set comprising at least one, 2, 3 genes, preferably 4, 5, 6, 7, 8, 9, 10, 11, 12, 13, 14, 15, 16, 17, 18, 19, 20, 21, 22, 23, 24, 25, 26, 27, 28, 29, 30, 35, 40, 50, 55, 60, 70, 80, 90 genes or specific portions thereof, primer sequence selected from the genes of Table 3 designated as “Up-regulated genes in grade 3 tumors”. Preferably, this gene set comprises at least 4 of these genes more preferably 4, 5, 6, 7 or 8 which are unexpectedly sufficient for obtaining an efficient prognosis and diagnosis of cancer especially breast cancer.

Preferably, these genes sets are proliferation related genes.

According to a first embodiment of the invention these genes are selected from the group consisting of UBE2C, KPNA2, TPX2, FOXM1, STK6, CCNA2, BIRC5, MYBL2. According to another embodiment of the present invention these genes are selected from the group consisting of the following proliferation related genes: CCNB1, CCNA2, CDC2, CDC20, MCM2, MYBL2, KPNA2 and STK6 preferably, the gene set comprising at least 4 genes, comprising at least 1 preferably at least 4 genes selected from the group consisting of CCNB1, CDC2, CDC20, MCM2, MYBL2 and KPNA2.

Preferably, the selection of at least 4 of the following genes, more preferably only these 4 genes (CCNB1, CDC2, CDC20 and MCM2 or more preferably only the 4 genes CDC2, CDC20, MYBL2 and KPNA2) are sufficient for obtaining an efficient prognosis and diagnosis of cancer especially breast cancer. The characteristics of the genes can be found in various databases, for instance upon the website www.genecards.org.

The preferred gene set comprises the gene CDC2, CDC20, MYBL2 and KPNA2. These genes present the following characteristics:

MYBL2: The protein encoded by this gene is a member of the MYB family of transcription factor genes, a nuclear protein involved in cell cycle progression. The encoded protein is phosphorylated by cyclin A/cyclin-dependent kinase 2 during the S-phase of the cell cycle and possesses both activator and repressor activities. It has been shown to activate the cell division cycle 2, cyclin D1, and insulin-like growth factor-binding protein 5 genes. Transcript variants may exist for this gene, but their full-length natures have not been determined.
KPNA2: Implicated in the import of protein to the nuclear envelope, KPNA2 is the regulator of cell cycle checkpoint mediators.
CDC2: The protein encoded by this gene is a member of the Ser/Thr protein kinase family. This protein is a catalytic subunit of the highly conserved protein kinase complex known as M-phase promoting factor (MPF), which is essential for G1/S and G2/M phase transitions of eukaryotic cell cycle. Mitotic cyclins stably associate with this protein and function as regulatory subunits. The kinase activity of this protein is controlled by cyclin accumulation and destruction through the cell cycle. The phosphorylation and dephosphorylation of this protein also play important regulatory roles in cell cycle control.
CDC20: Appears to act as a regulatory protein interacting with several other proteins at multiple points in the cell cycle. It is required for two microtubule-dependent processes, nuclear movement prior to anaphase and chromosome separation.

Advantageously, the kit according to the invention may further comprise the following primer sequence SEQ ID 1 to SEQ ID 16.

The kit or device according to the invention or the gene set according to the invention could also comprise additional normalization genes used as reference preferably, these genes are selected from the group consisting of the gene TFRC, GUS, RPLPO and TBP.

Advantageously, the primer sequence for the amplification of these genes are also present in the kit according to the invention preferably they have the sequence SEQ ID 17 to SEQ ID 24. These sequences are identified in the Table 4.

The kit or device according to the invention the tumor sample submitted through diagnosis is from a tissue affected by a cancer selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, or brain cancer. Preferably, this tumor sample is a breast tumor sample.

These genes set may also further comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 genes selected from the genes of Table 3 designated as “Up-regulated genes in grade 1 tumors”.

The gene sequences of this gene set can be bound to a solid support (micro-well plate, plates beads of glass or plastic material etc.) surface as an array and be present in a diagnostic kit or device, possibly including means for real time PCR analysis (preferably for qRT-PCR amplification).

The present invention is also related to the following primer sequences SEQ ID NO 1 to SEQ ID NO 16. For a specific amplification of these preferred 8 genes preferably present in the kit or device of the invention.

The kit or device according to the invention or the gene set according to the invention could also comprise additional normalization genes used as references. Preferably, these references genes are selected from the group consisting of the genes TFRC, GUS, RPLPO and TBP. Advantageously, the primer sequences SEQ ID NO 17 to SEQ ID NO 24 for the amplification of these reference genes are also present in the kit or device according to the invention. These primer sequences are identified in the Table 4. This kit or device may further comprise a computerized system comprising the gene sequence of this genes set bound upon a solid support surface as an array and a processor module, preferably configured to calculate gene expression grade index GGI or relapse score (RS) based on the gene expression and possibly to generate a risk assessment for a tumor sample. The present invention is also related to a method that allows a binding between nucleotide sequences obtained from a tumor sample one or more preferably 2, 3, 4, 5, 6, 7, 8, 9, 10, 11, 12, 13, 14, 15, 16, 17, 19, 20, 21, 22, 23, 24, 25, 26, 27, 28, 29, 30, 35, 40, 50, 55, 60, 70, 80, 90 genes or specific portion thereof selected from the genes of table 3 designated as “Up-regulated genes in grade 3 tumors” preferably at least the 8 or 4 genes above described more preferably CCNB1, CCNA2, CDC2, CDC20, MCM2, MYBL2, KPNA2 and STK6 more particularly CCNB1, CDC2, CDC20, MCM2 or CDC2, CDC20, MYBL2 and KPNA2 or the primer sequences SEQ.ID.NO.01 to SEQ.ID.NO.16 possibly combined with the primer sequences SEQ.ID.NO.17 to SEQ.ID.NO.24 for an amplification of these reference genes that are preferably present in the kit according to the invention for a prognosis or a diagnosis of cancer. Preferably, the method according to the invention is based upon genetic amplification, preferably a qRT-PCR based upon the use of the primer sequences above described which allows an amplification of the preferred genes of the gene set.

Another aspect of the present invention is related to the method comprising the steps of

(a) measuring gene expression in a tumor sample submitted to an analysis and obtained from a mammal subject, preferably a human patient;
(b) calculating the gene-expression grade index (or genomic grade) (GGI) of the tumor sample using the formula:

$${\sum }_{j ∈ {G}_{3}} {x}_{j} - {\sum }_{j ∈ {G}_{1}} {x}_{j}$$

wherein: x is the gene expression level of mRNA, G₁ and G₃ are sets of genes up-regulated in histological grade 1 (HG1) and histological grade 3 (HG3), respectively, and j refers to a probe or probe set wherein the gene set comprises or correspond (consist of) the gene set of the invention.

In the method, kit or device according to the invention, the tumor sample submitted to a diagnosis is (obtained) from a tissue affected by a cancer selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, or brain cancer. Preferably, this tumor sample is a breast tumor sample (more preferably a histological breast tumor sample grade HG2. The sample could be also frozen (FS) or dried tumor sample (paraffin-embedded tumor samples (FFPE)) of an (early breast cancer (BC)) patient.

This embodiment may further comprise designating the tumor sample as low risk (GG1) or high risk (GG3) based on the gene expression grade index (GGI). This embodiment may further comprise providing a breast cancer treatment regimen for a patient consistent with the low risk or high risk designation of the breast tumor sample submitted to the analysis.

The gene expression grade index GGI may include cutoff and scale values chosen so that the mean GGI of the HG1 cases is about −1 and the mean GGI of the HG3 cases is about +1. The cutoff value is required for calibration of the data obtained from different platforms applying different scales:

$$GGI = scale [ {\sum }_{j ∈ {G}_{3}} {x}_{j} - {\sum }_{j ∈ {G}_{1}} {x}_{j} - cutoff ]$$

The G₁ gene set may comprise at least one gene selected from the genes in Table 3 designated as “Up-regulated in grade 1 tumors”. Preferably, the G₁ gene set comprises at least 2, 3 of 4, 5, 6, 7, 8, 9, 10, 11, 12, 13, 14, 15, 16, 17, 18, 19, 20, 21, 22, 23, 24, 25, 26, 27, 28, 29, 30, 35, 40, 50 of these genes, and may include the entire set. The G₃ gene set may comprise at least one gene selected from the genes in Table 3 designated as “Up-regulated in grade 3 tumors.” Preferably, the G₃ gene set comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100 of those genes, and may include the entire set. Preferably the preferred gene set and the mentioned selected genes according to the invention above described.

In another aspect of the invention, the method according to the invention comprises the steps of

(a) measuring gene expression in a tumor sample;
(b) calculating a relapse score (RS) for the tumor sample using the formula:

$${\sum }_{i ∈ G} {w}_{i} {\sum }_{j ∈ {P}_{i}} \frac{{x}_{ij}}{{n}_{i}}$$

wherein: G is a gene set that is associated with distant recurrence of cancer, Pᵢ is the probe or probe set, i identifies the specific cluster or group of genes, wᵢ is the weight of the cluster i, j is the specific probe set value, xᵢⱼ is the intensity of the probe set j in cluster i, and nᵢ is the number of probe sets in cluster i.

This embodiment may further comprises the step of classifying the said tumor sample based on the relapse score as low risk or high risk for cancer relapse. The cutoff for distinguishing low risk from high risk may be a relapse score (RS) of from −100 to +100 or a relapse score (RS) of from −10 to +10. The relapse may be relapse after treatment with tamoxifen or other chemotherapy, endocrine therapy, antibody therapy or any other treatment method used by the person skilled in the art. Preferably, the relapse is after treatment with tamoxifen.

The patient's treatment regimen may be adjusted based on the tumor sample's cancer relapse risk status. For example (a) if the patient is classified as low risk, treating the low risk patient sequentially with tamoxifen and sequential aromatase inhibitors (AIs), or (b) if the patient is classified as high risk, treating the high risk patient with an alternative endocrine treatment other than tamoxifen. For a patient classified as high risk, the patient's treatment regimen may be adjusted to chemotherapy treatment or specific molecularly targeted anti-cancer therapies.

The gene set may be generated from an estrogen receptor (or another marker specific of the cancer tissue sample) positive population. The gene set may be generated by a variety of methods and the component genes may vary depending on the patient population and the specific disorder.

Another embodiment of the invention provides a computerized system or diagnostic device (or kit), comprising: (a) a bioassay module, preferably a bioarray, configured for detecting gene expression for a tumor sample based on the gene set of the invention; and (b) a processor module configured to calculate GGI or RS of the tumor sample based on the gene expression and to generate a risk assessment for the breast tumor sample. The bioassay module may include at least one gene chip (micro-array) comprising the gene set. The gene set may include at least one, 2 or 3 gene(s), preferably at least 4, 5, 6, 7, 8, 9, 10, 11, 12, 13, 14, 15, 16, 17, 18, 19, 20, 21, 22, 23, 24, 25, 26, 27, 28, 29, 30, 35, 40, 50 genes, selected from the genes in Table 3 designated as “Up-regulated in grade 1 tumors” or may include the entire set. The gene may include 1, 2 or 3 genes preferably at least 4 5, 6, 7, 8, 9, 10, 11, 12, 13, 14, 15, 16, 17, 18, 19, 20, 21, 22, 23, 24, 25, 26, 27, 28, 29, 30, 35, 40, 50, 55, 60, 65, 70, 75, 80, 85, 90 genes selected from the genes in Table 3 designated as “Up-regulated in grade 3 tumors” or may include the entire set.

The inventors have also observed unexpectedly that it is possible to use the primer(s) according to the invention for obtaining an efficient qRT-PCR assay upon a tumor sample obtained directly from a mammal (including a human patient) or upon conserved sample especially frozen (FS) and dried tumor sample (paraffin-embedded tumor samples (FFPE)) from early breast cancer (BC) patient.

The inventors have tested such qRT-PCR assay accuracy and concordance with original micro-array derives GGI (Genomic Grade Index) using breast cancer population from which frozen and paraffin-embedded tumor samples tissues were collected and inventors have obtained a statistical significant correlation between the Genomic Grade Index (GGI) generated by micro-array and these qRT-PCR assay using frozen (FS material) as well as paraffin-embedded samples (FFPE material) and between the Genomic Grade Index (GGI) using qRT-PCR derived from frozen (FS) and paraffin-embedded tumors samples (FFPE).

The inventors have tested the prognostic value on an independent ER-positive tamoxifen only treated frozen breast cancer population and on an independent population of paraffin-embedded breast cancer samples consecutively diagnosed at Jules Bordet Institute.

The inventors have observed unexpectedly that a high Genomic Grade Index (GGI) levels assessed by qRT-PCR associated with a higher risk of recurrence in the global breast cancer population and particularly in the ER-positive patients. This was in accordance with the present micro-array result. In multivariate analyses, the GGI assessed by qRT-PCR remained significant. Therefore, qRT-PCR based on a limited number of genes, preferably the gene selected in the gene set according to the invention, recapitulate in an accurate and reproducible manner the prognostic power of Genomic Grade Index derived from micro-array using both frozen and paraffin-embedded tumor samples (FFPE).

Another aspect of the present invention concerns a method for an efficient screening and/or testing of active compound(s) (or treatment method based upon an administration of active compounds) upon cancer that comprises the method and tools according to the invention especially that comprises the step of testing and monitoring and modulating the effects of this compound upon a tumor sample of a mammal subjects including human patients by testing the risk of a cancer in these subjects with the method and tools of the invention before and after this compound is applied to the patient.

Therefore, this method comprises a selection of one or more active compounds which could be administrated separately or simultaneously to a mammal subject for treating or preventing a cancer testing the efficacy of said active compound(s) by collecting from the treated mammal a tumor sample (biopsy) before and after the administration of said compound(s) to the mammal, submitting said tumor sample to a diagnosis with the method and tools according to the invention (by detecting gene expression in said tumor sample with the genes set according to the invention or the kit or device according to the invention), possibly generating a risk assessment of this tumor sample before or after the administration of the tested compounds and possibly identifying if the compound(s) may have an effect upon a cancer or may present a risk of developing a cancer. Consequently, this method could be a screening testing or monitoring method of new antitumoral compounds.

The method according to the invention could be applied upon a mammal presenting a predisposition to a cancer or subject, including a human patient suffering from cancer for the monitoring of the effect of the therapeutical active compounds.

### BRIEF DESCRIPTION OF THE DRAWINGS

FIG. 1 is representing heatmaps showing the pattern of gene expression in the training (panel a) and the validation sets (panel b). The horizontal axis corresponds to the tumors sorted first by HG and then by GGI as the secondary criterion. The vertical axis corresponds to the genes. The GGI values of each tumor and the relapse free survival are indicated underneath. Two groups of genes are found: those that are highly expressed in grade 1 (16 probe sets; highlighted in red) and, reciprocally, those highly expressed in grade 3 (112 probe sets). The GGI values for HG2 tumors cover the range of values for HG1 and HG3, and those with high GGI tend to relapse earlier (red dots).

FIG. 2 shows Kaplan-Meier RFS analysis based on the HG (panel a) and the GG (panel b) for data pooled from the validation datasets 2-5 (table 1). HG1, HG2 and HG3 can be split further into low and high risk subsets by GG, indicating that GG is an improvement over HG (panel c, d and e respectively). ER status identifies some, but not all, of the patients with poor prognosis (panel f).

FIG. 3 shows Kaplan-Meier RFS analysis based on the NPI (a) and the NPI-GG (b) classification. NPI-GG improves the prognostic discrimination in both low (panel c) and high (panel d) risk NPI subsets, but not vice versa (panels e and f). The Sorlie et al. dataset was excluded from this analysis because of incomplete tumor size information.

FIG. 4 shows a Forest plot for hazard ratios for HG2 patients split into GG1 and GG3, showing consistent results in different datasets Hazard ratios were estimated with Cox proportional hazard regressions, horizontal lines are 95% confidence intervals for the hazard ratio. P values were determined by the log rank test.

FIG. 5 shows distant metastasis free survival (DMFS) analysis based on the 70-gene expression signature (left row, panels a, c and e) and on GGI (right row, panels b, d and f) for data from the Van de Vijver et al. validation study. a) and b) are all patients, c) and d) are node-negative, and e) and f) are node-positive patients. Note that the node-negative subset includes patients used to derive the 70-gene signature.

FIG. 6 represents a genomic grade applied to previously reported molecular subtypes.

FIG. 7 represents Kaplan Meyer survival curves for distant metastasis free survival for GGI (high vs. low).

FIG. 8 represents survival analyses in function of index defined by qRT-PCR performed with the 4 selected genes according to the invention.

FIG. 9 represents survival analyses in function of index defined by micro-array.

FIG. 10 represents survival analyses of patient ER+ in function of index defined by qRT-PCR performed with the 8 selected genes.

FIG. 11 represents survival analyses of patient ER+ in function of the index defined by qRT-PCR assay based upon the 4 selected genes according to the invention.

### DETAILED DESCRIPTION OF THE INVENTION

### Definitions

Most terms scientific, medical and technical terms are commonly understood to one skilled in the art.

The term “micro-array” refers to an ordered arrangement of hybridizable array elements, preferably polynucleotide probes, on a substrate (an insoluble solid support).

The terms “differentially expressed gene”, “differential gene expression” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

Gene expression profiling: includes all methods of quantification of mRNA and/or protein levels in a biological sample.

The term “prognosis” is used herein to refer to the prediction of the likelihood of cancer-attributable death or progression, including recurrence, metastatic spread, and drug resistance, of a neoplastic disease, such as breast cancer.

The term “prediction” is used herein to refer to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention are valuable tools in predicting if a patient is likely to respond favorably to a treatment regimen, such as, chemotherapy with a given drug or drug combination, and/or radiation therapy, or whether long-term survival of the patient, following surgery and/or termination of chemotherapy or other treatment modalities is likely.

The term “high risk” means the patient is expected to have a distant relapse in less than 5 years, preferably in less than 3 years.

The term “low risk” means the patient is expected to have a distant relapse after 5 years, preferably in less than 3 years.

The term “tumor sample” corresponds to any sample obtained from a tissue or cell mammal subject (preferably a human patient that may present a predisposition to a cancer) and obtained from a biological fluid of a mammal subject (preferably a human patient) or a biopsy, including frozen or dried (paraffin embedded tumor sample, preferably human) tumor sample.

The term “tumor,” as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

Raw “GGI” (Gene expression grade index) is the sum of the log expression (or log ratio) of all genes high-in-HG3-sum of the log expression (or log ratio) of all genes high-in-HG1 and can be written as:

$${\sum }_{j ∈ {G}_{3}} {x}_{j} - {\sum }_{j ∈ {G}_{1}} {x}_{j}$$

wherein:
x is the gene expression level of mRNA,

G₁ and G₃ are sets of genes up-regulated in HG1 and HG3, respectively, and j refers to a probe or probe set.

GGI may include cutoff and scale values chosen so that the mean GGI of the HG1 cases is about −1 and the mean GGI of the HG3 cases is about +1:

$$GGI = scale [ {\sum }_{j ∈ {G}_{3}} {x}_{j} - {\sum }_{j ∈ {G}_{1}} {x}_{j} - cutoff ]$$

The cutoff in GGI is 0 and corresponds to the mean of means.
GGI ranges in value from −4 to +4.

### Example 1

### Material and Methods for Development of Grade Index (GGI) Patient Demographics

Six datasets of primary breast cancer were used, four of which were publicly available (Table 1). No patient received adjuvant chemotherapy and some had received adjuvant tamoxifen treatment. Histological grade (HG) was based on the Elston-Ellis grading system. Each institutional ethics board approved the use of the tissue material.

TABLE 1
Microarray datasets used in this study

| | | | | Microarray | Systemic | Refer- |
|---|---|---|---|---|---|---|
| Identifier | | Institution | N | Platform | Treatment | ence |
| 1. | Training | Karolinska | 24 | Affymetrix | yes | this |
| | set | John | 40 | U133A | (tamoxifen | paper |
| | (KJX64) | Radcliffe | | | only) | |
| 2. | Validation | Karolinska | 68 | Affymetrix | No | this |
| | set | John | 61 | U133A | | paper |
| | (KJ129) | Radcliffe | | | | |
| 3. | Sotiriou et | John | 99 | cDNA | Yes | 10 |
| | al. (NCI) | Radcliffe | | (NCI) | | |
| 4. | Sorlie et | Stanford | 80 | cDNA | Yes | 11 |
| | al. | | | (Stanford) | | |
| | (STNO) | | | | | |
| 5. | van't Veer | Netherlands | 97 | Agilent | No | 4 |
| | et al. | Cancer | | | | |
| | (NKI) | Institute | | | | |
| 6. | Van de | Netherlands | 295 | Agilent | No | 5 |
| | Vijver | Cancer | [61 | | | |
| | et al. | Institute | also | | | |
| | (NKI2) | | in 5)] | | | |
| | Total | | 703 | | | |

The samples from Oxford were processed at the Jules Bordet Institute in Brussels, Belgium, and those from Sweden at the Genome Institute of Singapore in Singapore. RNA extraction, amplification, hybridization and scanning were done according to standard Affymetrix protocols. Affymetrix U133A Genechips (Affymetrix, Santa Clara, Calif.). Gene expression values from the CEL files were normalized using RMA (12).

The default options (with background correction and quantile normalization) were used. The output were in logarithmic scale.

The normalizations were done separately for .CEL files from different institutions and batch of measurements. In subsequent analysis, the expression data matrices were treated as if they were “blocks” of separate studies. The training set KJX64 consisted of two blocks (corresponding to two different institutions), and so did the validation set KJ129.

STNO The Stanford/Norway dataset (Sorlie et al., 2001) was downloaded from http://genome-www.stanford.edu/breast.cancer/mopo.clinical/data.shtml

It consists of 85 arrays, with several different chip designs. Only the probes that are common to all were used. The gene expression values used are from the column LOG RAT2N MEAN in the array data files. No further transformation is applied prior to computing the GGI. When more than one spot corresponds to a probe, their average was used.

All 85 patients were used in the heatmap, but only those with non missing and non zero follow up time were used in survival analysis. This dataset was excluded from analysis involving tumor size, since this information was not available (Only TNM category was given, but the conversion to tumor size is not straightforward, particularly when one is concerned with what is appropriate for the NPI formula).

NKI/NKI2 The data set NKI (van't Veer et al., 2002) and NKI2 (van de Vijver et al., 2002) were downloaded from Rosetta website www.rii.com. The log ratio was used without further transformation. For NKI2, flagged expression values were considered missing. Age, tumor size, and histological grade were not available for NKI2.

The field ‘conservFlag’ in the clinical data table were used to stratify the dataset into two groups. Each group had its own threshold for deciding ‘good’ vs ‘poor’ prognosis, as was done for in the original results in van de Vijver et al. (2002).

NCI This dataset from Sotiriou et al. (2003) was downloaded from the PNAS web site http://www.pnas.org/cgi/content/full/1732912100/DC1. The expression values were not modified.

### Statistical Analysis

Gene selection was done only on the KJX64 dataset, which are all estrogen receptor (ER)-positive and either HG1 or HG3. Dataset KJ129 (43 ER-negative, all node-negative, no systemic treatment) was used as the validation set, along with other previously published data (see table 1). ER-positive tumor s were used for the training set, because ER-status and grade were not independent, with very few ER-negative, HG1 tumor s. Using all HG1 and HG3 tumor s regardless of the ER status would have resulted in spurious associations.

The standardized mean difference of Hedges and Olkin (13), was used to rank genes based on their differential expression with respect to HG1 or HG3. This meta-analytical score is similar to the t-statistic, but better suited for our training set which consisted of array data originating from two different centres.

To control for multiple testing, the maxT algorithm of Westfall and Young (14), with an extension proposed by Korn et al. (15), was applied to compute false discovery counts (FDC). All 22,283 probe sets were considered. Probe sets having a family-wise error rate p-value lower than 0.05 with FDC>2 were identified. Mapping of probes between platforms was done through Unigene (build #180), according to the method in Praz et al. (16).

The gene-expression grade index (GGI) is defined as:

$$GGI = scale [ {\sum }_{j ∈ {G}_{3}} {x}_{j} - {\sum }_{j ∈ {G}_{1}} {x}_{j} - cutoff ]$$

where x is the logarithmic gene expression measure, and G₁ and G₃ are the sets of genes up-regulated in HG3 and HG1, respectively. These sets differed across platforms. For convenience, the cutoff and the scale were chosen so that the mean GGI of the HG1 cases was −1 and that of the HG3 cases was +1. This rescaling was done separately for each data source.

The Nottingham Prognostic Index (NPI) was calculated according to Todd et al. (17):

NPI=0.2×size [cm]+lymph node status+histological grade.

An index called NPI/GG was defined, where HG was replaced by GG. Cases with NPI≧3.4 to be high risk in both NPI and NPI/GG were considered. Survival data were visualized using Kaplan-Meier plot. The hazard ratios (HR) were estimated using Cox regression, stratified by the data source. Assumption-free comparisons were done using the stratified log rank test.

### Heat Maps

For visualization, the values used in the heatmaps for each probe were meancentered across patients. No genespecific scaling (standardization) was done, in order to keep the information about the relative signal strength of all probes. The color tone was calibrated such that saturated red and green were reached at the values three times the standard deviation of the expression values of the entire matrix. Note that the scaled GGI values were not affected by genespecific centering.

### Survival Analysis

The survival package for R was used by Terry Therneau and a custom program for the KaplanMeier plots, which was checked against the output of the survival package for correctness.

### Mapping Across Microarray Platforms

The approach of CleanEx database (http://www.cleanex.isbsib.ch), described in Praz et al. (2004) was used. Probe identifies were first mapped into sequence accession number. Unigene (build 180) were then used to map the correspondence between platforms. For Affymetrix chips, probesets which contain oligos that were ambiguously mapped to more than one Unigene id were excluded.

### Results

### Differentially Expressed Genes Between High and Low Grade Subsets

242 Probe sets corresponding to 183 unique genes with FDC>2 at family-wise error rate p-value of 0.05, corresponding to a low false discovery proportion of 0.008 were identified (Table 3). Of these, a list of 128 probe sets (97 genes) based on a more conservative criterion (FDC>0 at p-value of 0.05) was used in all subsequent analyses, except for checking common genes with signatures published by others, where we used the 183-gene list.

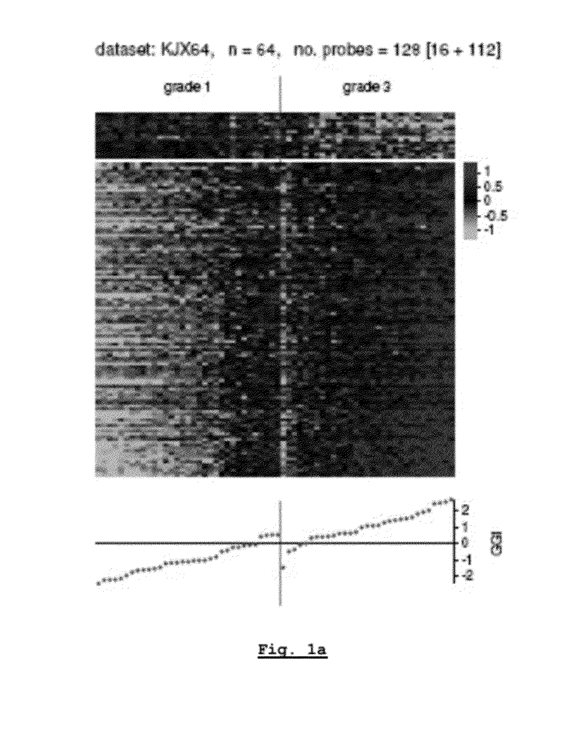

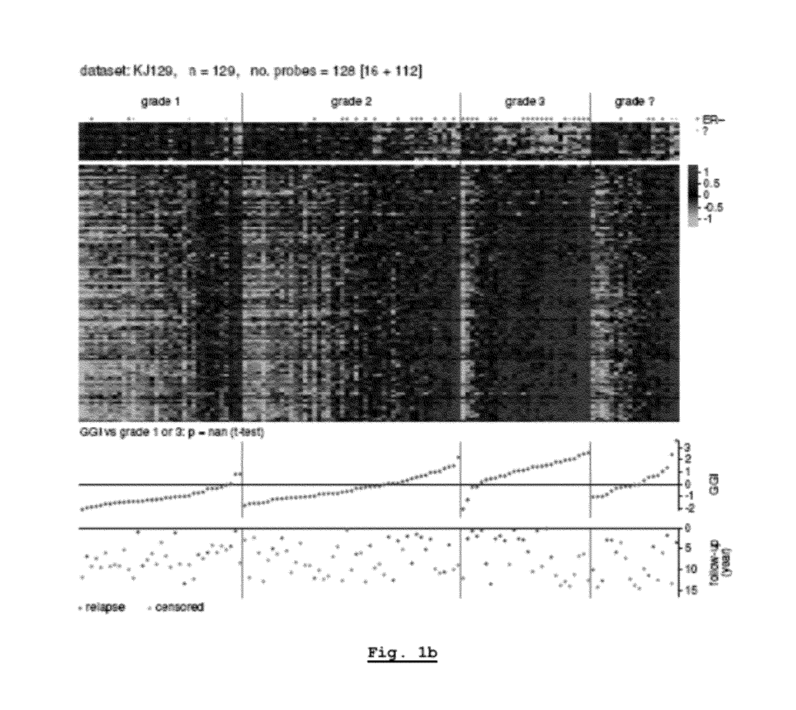

FIG. 1a shows two strong and reciprocal patterns of expression clearly associated with HG1 and HG3. Many genes up-regulated in HG3 were mostly associated with cell cycle progression and proliferation (Table 3). The same gene selection algorithm to contrast HG2 tumors with a pool combining HG1 and HG3 tumors were applied. This yielded no differentially expressed genes. Thus, the HG2 population as a whole has no peculiar characteristics of its own that are independent from the HG1 and HG3 distinction.

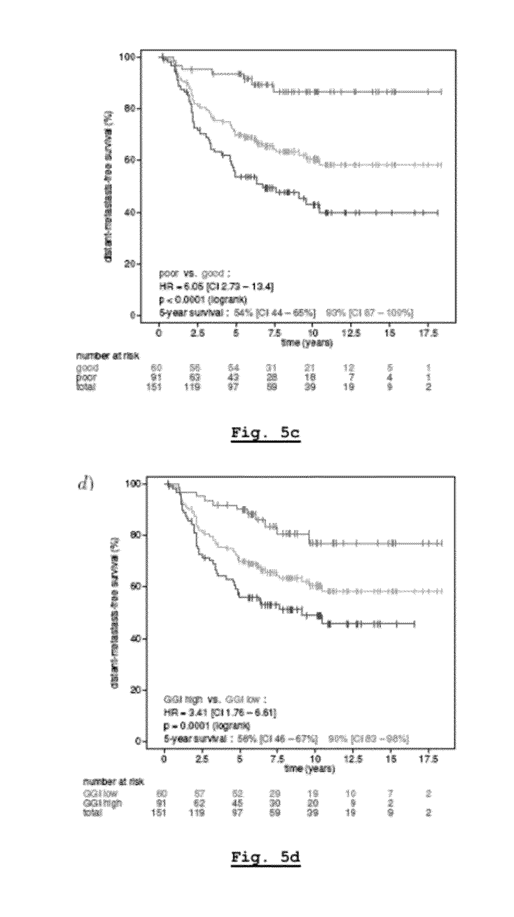

The list of 128 probe sets was then applied to untreated breast cancer patients (dataset KJ129). As shown in FIG. 1b, visual inspection revealed an expression pattern for HG1 and HG3 similar to that which was observed on the training set (FIG. 1a). The GEP of the grade 2 population looked like a mixture of grade 1 and grade 3 cases, rather than intermediate between the two. To make this observation more objective, the GGI (which essentially summarizes the differences in the GEP of the reporting genes by averaging their expression levels) was defined. As shown under the heat maps in FIG. 1, the GGI distribution of HG2 covered the range of the GGI values of HG1 and HG3, confirming the visual impression. A similar observation was made on the three previously published datasets, despite differences in the clinical populations and micro-array platforms (see FIGS. 6a, b, and c).

### Histological Grade, Gene-Expression Grade (GG) and Prognosis

These findings lead to showing that intermediate histological grade can be replaced by low and high grade based on gene expression. Gene-expression grade (GG) based on the GGI score was defined. Patients were classified as GG1 (low grade) if their GGI value was negative or as GG3 (high grade) otherwise. Note that the GGI score of zero corresponds to the midpoint between the average GGI values of HG1 and HG3 (see methods). This choice might not be clinically optimal and could be improved based on the trade-off between the cost of treatment and risk, but it would be sufficient for evaluating the prognostic value of GGI.

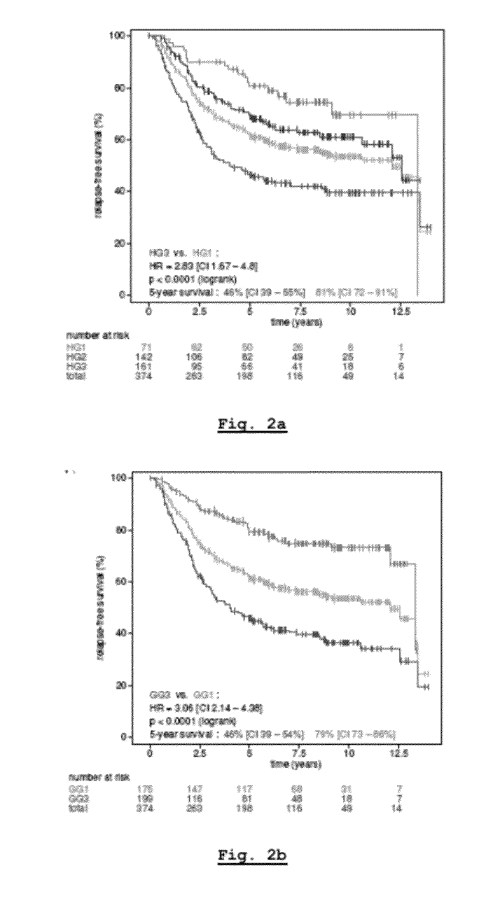

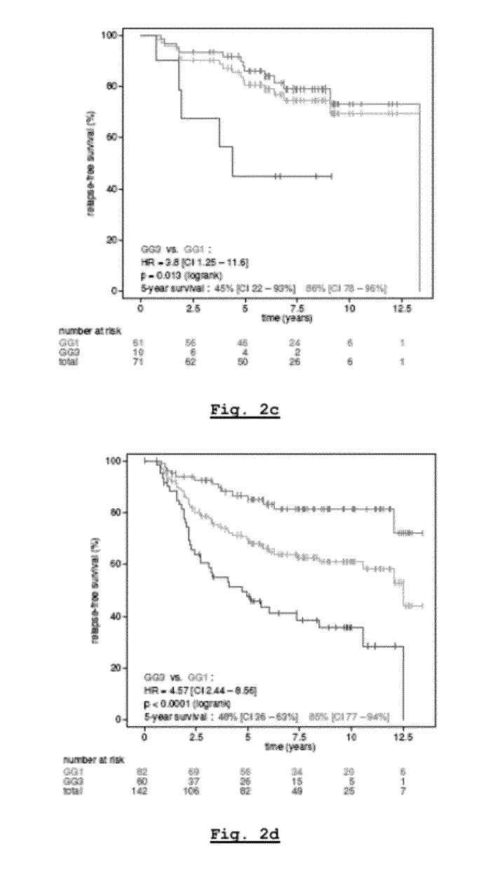

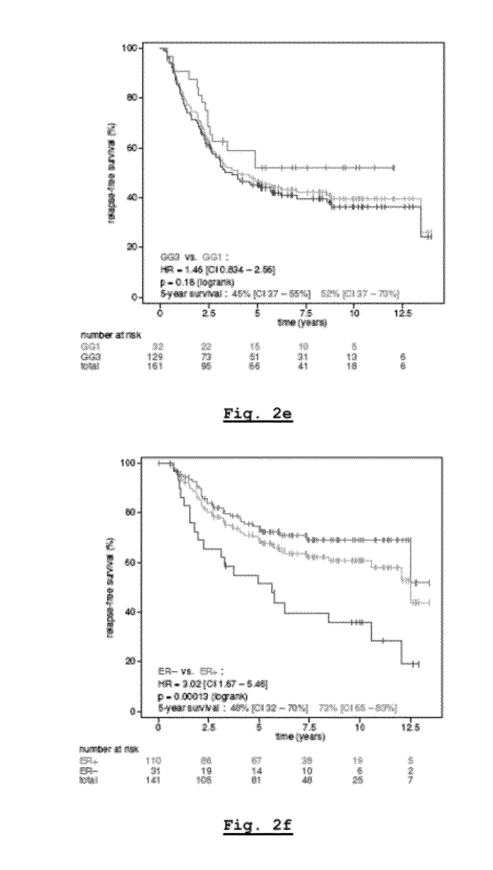

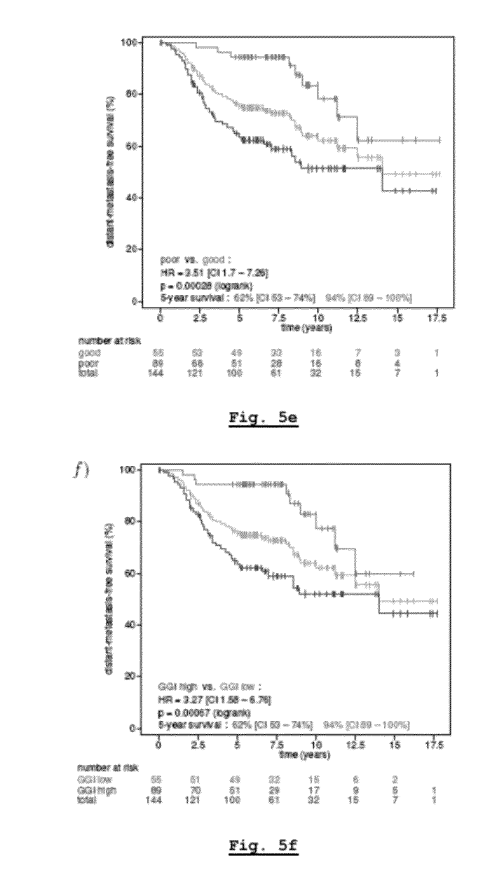

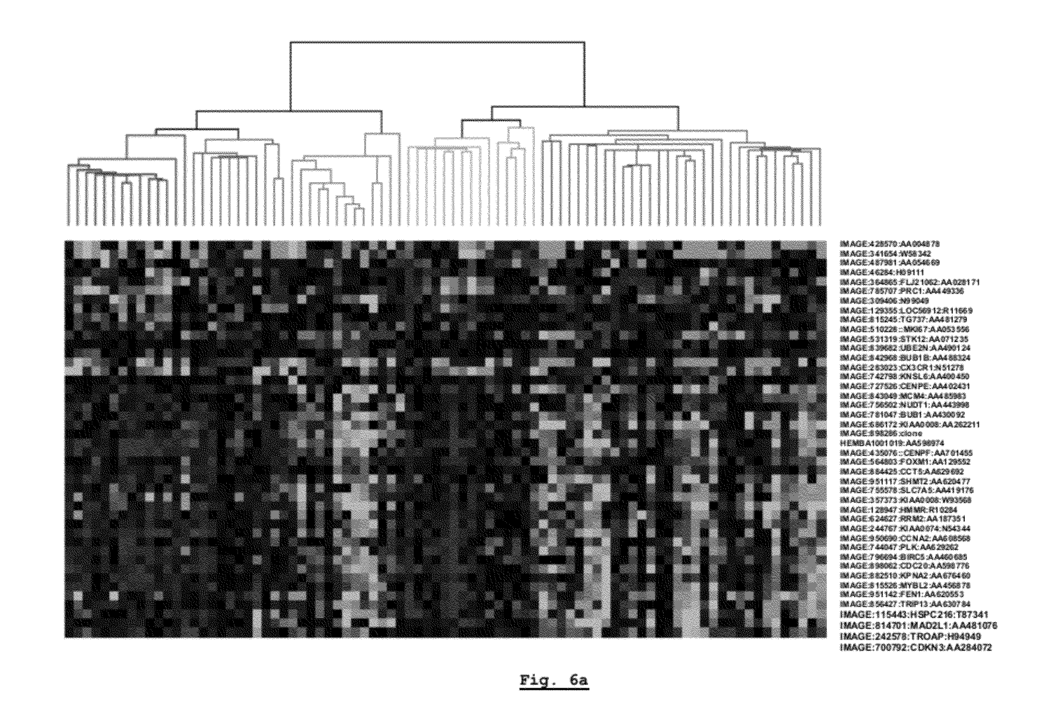

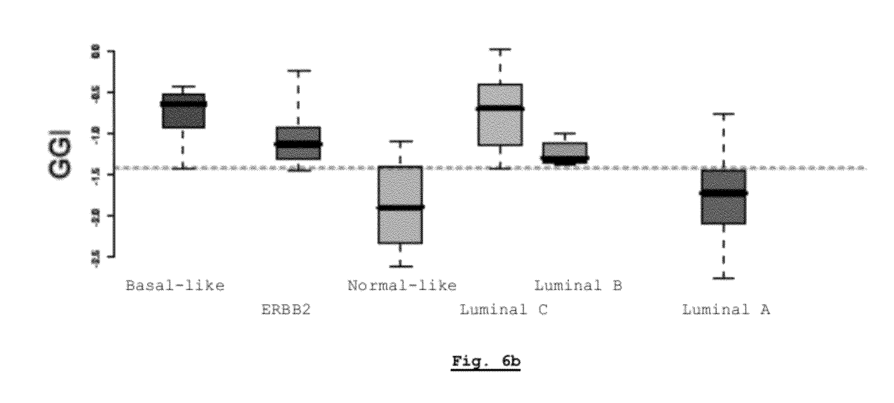

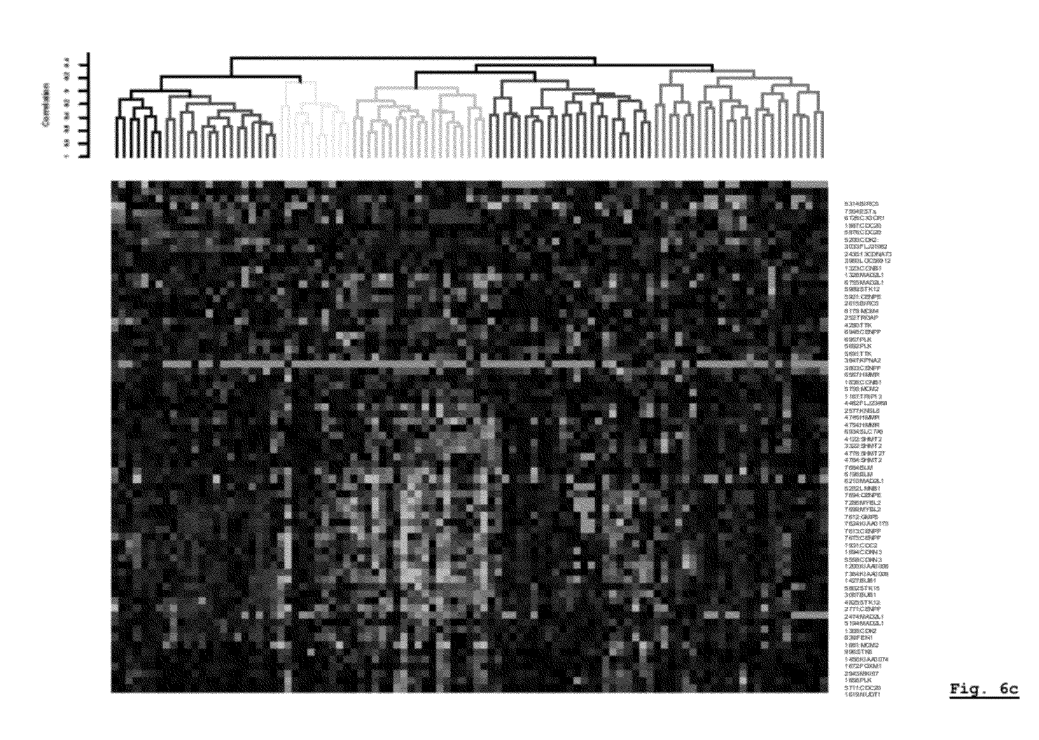

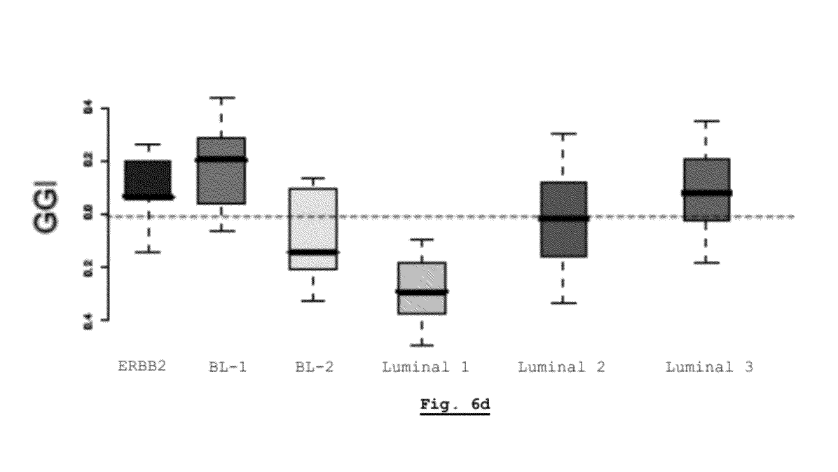

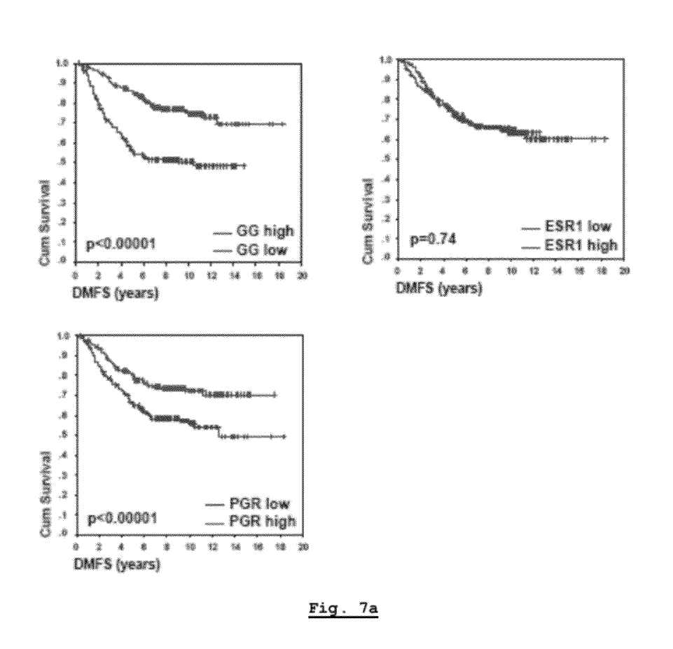

For this purpose, breast cancer samples derived from a pool of our own validation population (KJ129) and additional datasets STNO, NCI and NKI (table 1) were used. In FIG. 2a, the association between histological grade and relapse-free survival (RFS) was examined. As expected, HG3 tumors had significantly worse RFS than HG1, while HG2 tumors had an intermediate risk and constituted 38% of the population. In FIG. 2b, GG1 and GG3 subgroups showed distinct RFS, similar to the RFS of HG1 and HG3 tumors, respectively. To examine how the discordance between GG and HG are related to prognosis, GG was split for each of the histological categories (FIGS. 2c, 2d and 2e). The most striking result was that GG split HG2 into two groups, namely HG2/GG1 and HG2/GG3, whose RFS were also respectively similar to those of HG1 and HG3 (FIG. 2d). The log rank test failed to reveal any significant difference in survival between HG1 and HG2/GG1, as well as between HG3 and HG2/GG3 (see FIG. 7). For comparison, ER status also had prognostic power in HG2 tumor s (FIG. 2f), although the hazard ratio was less than that of GG (FIG. 2d). Notably, the ER-positive group showed similar RFS as the total population.

While GG was better than HG by classifying some patients with poor prognosis in the HG1 population (FIG. 2c), the reverse seems to be the case in HG3 population: it classified some patients as low-risk despite their poor prognosis (FIG. 2d). Thus, in the case of discordance involving low and high grade categories, neither GG nor HG were consistently outperform the other. It seemed that whichever decided to classify as high grade tended to be more accurate prognostically. This suggests that for both HG and GG, correctly detecting any indication of high grade was easier than accurately declaring it absent. If this observation is confirmed by future studies, corrections should be done in clinical practice, for example by using a rule which substitutes HG1 and HG2, but not HG3, by GG. However, the frequency of this type of discordance in the data used here was relatively small and such modifications were not used in this study, which aims to characterize GG purely on its own.

TABLE 2
Multivariate analysis of breast cancer prognostic factors (N = 302)

| | Univariate analysis | | Multivariate analysis | |
|---|---|---|---|---|
| | Hazard ratio | | Hazard ratio | |
| | (95% CI) | p | (95% CI) | p |
| Gene- | | | | |
| Expression | | | | |
| Grade | | | | |
| GG3 vs GG1 | 2.97 (2.03-4.37) | 0.0001 | 2.29 (1.44-3.63) | 0.0004 |
| Histological | | | | |
| Grade | | | | |
| 2 + 3 vs 1 | 1.93 (1.15-3.28) | 0.0150 | 0.85 (0.46-1.57) | 0.61 |
| 3 vs 1 + 2 | 2.03 (1.41-2.92) | 0.0001 | 1.25 (0.80-1.94) | 0.33 |
| Estrogen | | | | |
| Receptor | | | | |
| Negative vs | 1.76 (1.24-2.49) | 0.0016 | 1.19 (0.81-1.76) | 0.38 |
| Positive | | | | |
| Nodal Status | | | | |
| Positive vs | 2.53 (1.34-4.78) | 0.0040 | 1.95 (1.01-3.73) | 0.045 |
| Negative | | | | |
| Tumor Size | | | | |
| >2 cm vs | 2.06 (1.41-3.03) | 0.0002 | 1.63 (1.10-2.43) | 0.015 |
| ≦2 cm | | | | |
| Age (years) | | | | |
| ≦50 vs >50 | 0.99 (0.69-1.42) | 0.97 | 1.13 (0.78-1.63) | 0.53 |

### Prognostic Value of GG in Multivariate Model

Almost all clinicopathological variables were significantly associated with clinical outcome in univariate analysis (Table 2). GG and HG status had the strongest effect. However, in multivariate analysis, only GG, nodal status and tumor size kept their significance, with GG having the largest hazard ratio. In accordance with FIG. 2, GG replaced HG when both were considered, and GG considerably reduced the prognostic impact of ER.

### GG and the Nottingham Prognostic Index

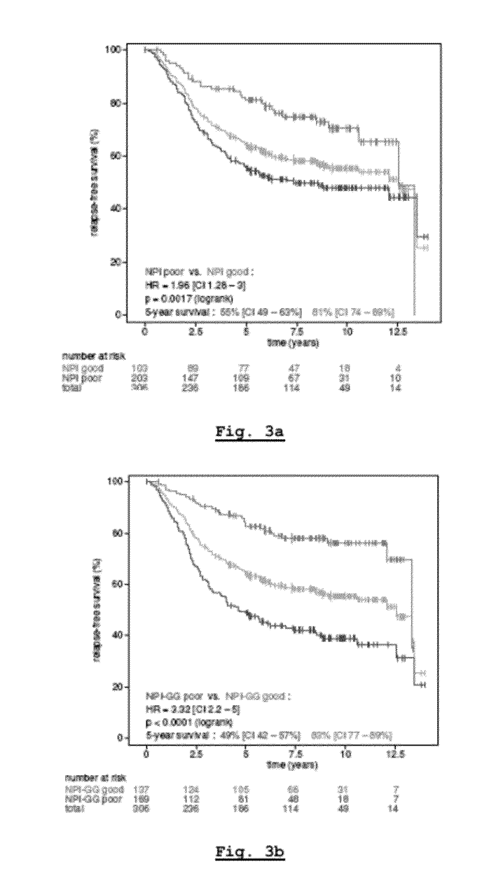

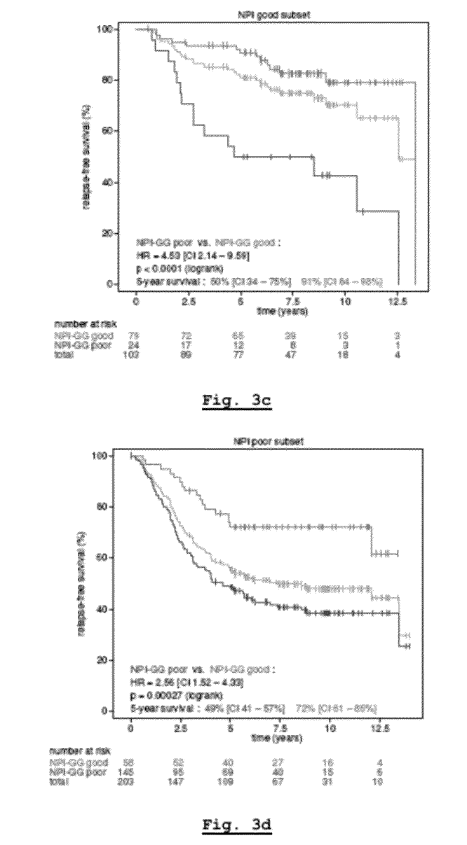

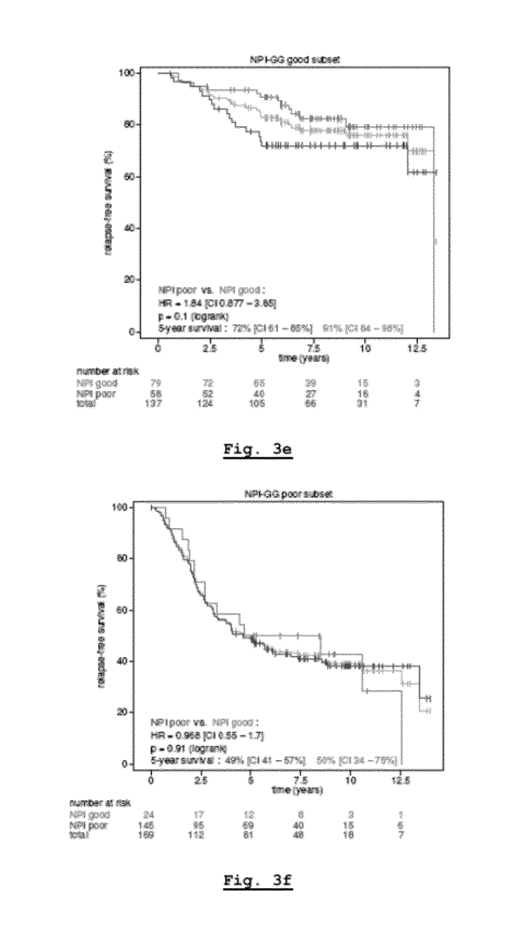

The independence of GG, nodal status and tumor size in explaining the disease outcome mirrored the Nottingham Prognostic Index (NPI), which combines HG, nodal status and size. To test whether GG can be used to improve this well-characterized risk score, we propose a score called NPI/GG, which is analogous to NPI except that HG is replaced by GG, with only two possible values (either 1 or 3). As shown in FIGS. 3a and 3b, NPI/GG was significantly more discriminative than classical NPI. Moreover, NPI/GG was able to split both the NPI low and high risk groups into subgroups with significantly different clinical outcome (FIG. 3c, 3d), while the reverse was not true (FIG. 3e, 3f).

### Example 2

### Consistent Prognostic Value of GG in Different Populations and Microarray Platforms

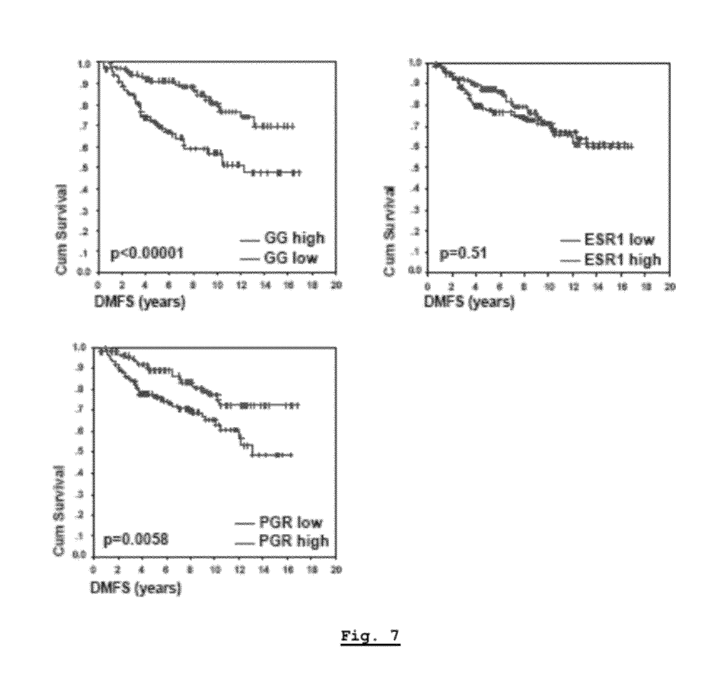

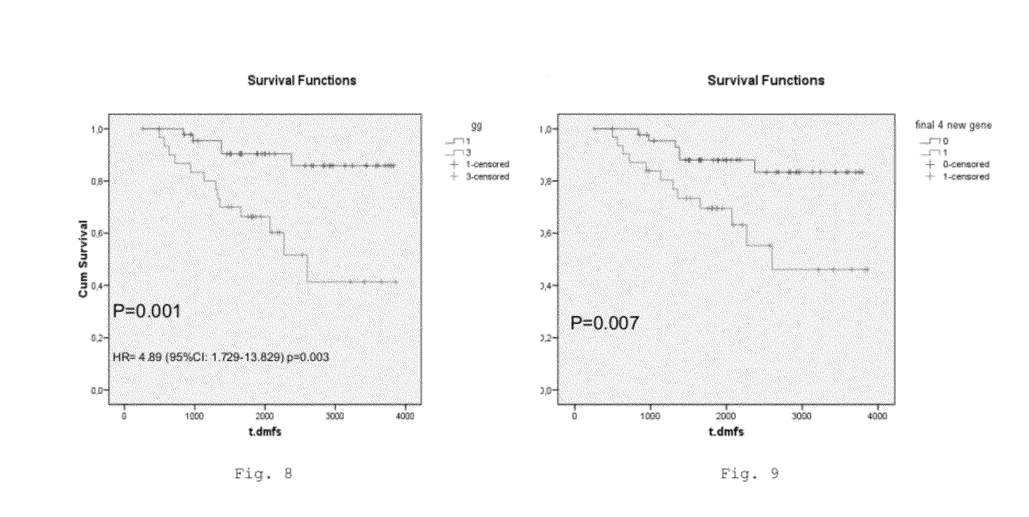

The results of the pooled analysis above were consistently present in the individual datasets, as shown by the forest plot of hazard ratios in FIG. 4. More complete results are shown in FIG. 8. FIG. 4 shows that in each independent validation dataset, GG divided the grade 2 populations into two distinct groups with statistically different clinical outcomes. There was no significant heterogeneity between the hazard ratios, even though the different datasets included heterogeneous patient populations, were graded by various pathologists and used different micro-array platforms.

Relationship with the 70-Gene Signature

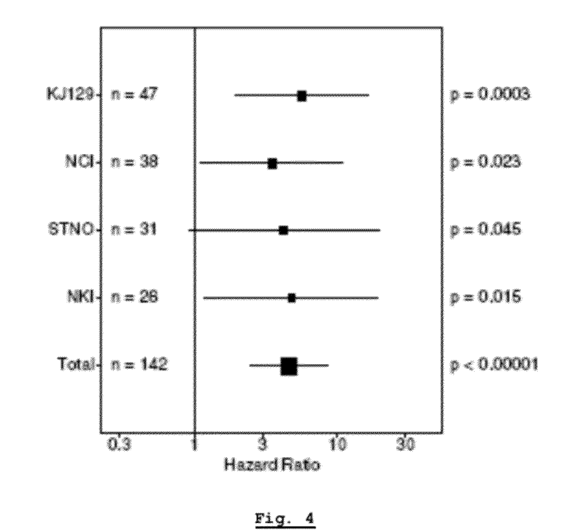

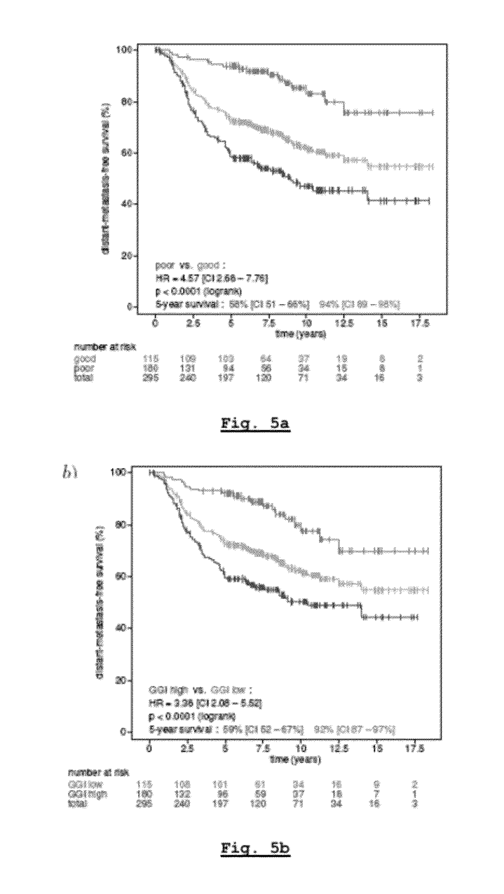

In their pioneering work, van't Veer et al. identified a 70-gene expression signature significantly correlated with distant metastasis in node negative breast cancer patients. The present list of 97 genes (128 probe sets) could be mapped to 93 genes (113 probes) in their Agilent arrays. To allow comparison under the same trade-off between risk and the cost of treatment as the Netherlands Cancer Institute (NKI) classification, cutoffs for GGI that gave the same numbers of patients in high- and low-risk groups were selected (see methods). FIG. 5 shows the comparisons between the NKI prognostic signature and the GGI on distant-metastases-free survival for the overall population (FIG. 5a, b), as well as for the node negative (FIG. 5c, d) and positive subgroups (FIG. 5e, f). Despite the fact that our probes were selected without using clinical outcome and had to be mapped across platforms, the results were strikingly close. Similar results were found when considering overall survival (see FIG. 9). Data were unavailable to compare relapse-free survival.

Low and high grade breast cancers were unexpectedly associated with many differentially expressed genes, the majority being involved in cell cycle and proliferation. For these genes, HG2 tumor s had heterogeneous transcriptional profiles that covered the range of variation of HG1 and HG3 tumor s. A similar observation was made in at least one previous report (18). Here, the clinical implications of this finding and discovered that the grade-related GEPs were also correlated with disease outcome are investigated.

As demonstrated by FIG. 4 improvements by GG were consistent across the different datasets which would have not been the case if the grading quality differed significantly between these studies. Similarly, FIG. 2a shows good prognostic separation between HG1 and HG3, indicating that the histological grading was of high quality. Furthermore, central pathologist review would still result in a significant portion of tumor s being classified as HG2. Finally, these results were more reflective of clinical reality, since grading by a central pathologist is rarely done in practice.

The approach in identifying GEP associated with prognosis is quite different from that used by other investigators. Instead of selecting the prognostic genes directly through their correlation with survival, one may identify them indirectly through histological grade, a well-established prognostic factor rooted in cell biology. This may explain the robustness and reproducibility of GGI across independent and heterogeneous validation sets and different micro-array platforms. Furthermore, since the GGI can be interpreted as “molecular grade”, it can be integrated easily into existing prognostic systems which uses histological grade, such as the NPI.

This gene selection process was not meant to define a specific set of genes to be used as a prognostic “signature”. The present invention aims to build a comprehensive “catalogue” where different sets of signatures could be chosen from. This was illustrated by the cross-platform applicability of the catalogue. Although the actual sets of probes used in various platforms differed in numbers and gene compositions, the results were still reproducible. It is remarkable to obtain good prognostic discrimination in very different datasets with a linear classifier where the weights of the genes were simply +1 or −1, based on their association with grade on a training set of 64 patients. Thus, the “grade signal” identified was not bound to a particular set of genes nor to any special combination of their expression levels, since the genes were highly correlated and the GGI effectively behaves as a single prognostic factor. It is still beneficial to use many genes, if only to provide redundancy against noise. The consequence for the development of practical diagnostic systems is that arbitrary subsets of the “grade gene catalogue” of the invention might be used, constrained only by technical considerations.

Jenssen and Hovig recently discussed two issues regarding the use of gene-expression signatures for prognosis. These were 1) the lack of agreement between genes included in different signatures and 2) the difficulty in understanding the biological basis of the correlation between the signatures and survival. The present gene catalogue is rich in genes with likely roles in cell cycle progression and proliferation. This class of genes is one important—if not the most important—component of any existing profile-based risk prediction method for breast cancer. In Paik et al., the “proliferation set”, whose five genes are all in our 183-gene catalogue (Table 3), was the one that had the largest hazard ratios in their extensive training and validation sets and has the highest weight in the “recurrence score” formula. The application to the NKI data in FIG. 5 also lends support to the idea that grade-related genes may constitute a significant portion of the prognostic power of the NKI 70-gene signature. When compared against our 183-gene catalogue, the following numbers of genes in common with other prognostic signatures: 11/70 and 30/231 genes (van't Veer et al.), 5/15 (Paik et al) and 7/76 (Wang et al.) were found.

In summary, gene-expression based grading could significantly improve current grading systems for the prognostic assessment of cancer, in particular breast cancer.

Reproduction of these findings across multiple independent datasets and across different platforms suggests our conclusions are robust. The GGI score does not require a specific set of genes nor is it bound to a particular detection platform. Grading based on the GGI can be incorporated into existing prognostic systems, by substituting HG with GG. Refined grading based on gene expression measurements could have important clinical application for breast cancer management in the future.

### Example 3

### Definition of Clinically Distinct Subtypes within Estrogen Receptor Positive Breast Carcinoma

### Materials and Methods

### Tumor Samples

Three hundred and thirty five early-stage breast carcinoma samples comprised our own dataset. Eighty-six of these samples have been previously used in another study and the raw data are available at the Gee Expression Omnibus repository database (http://www.ncbi.nlm.nih.gov/geo), with accession code GSE2990. These samples had received no adjuvant systemic therapy. Two hundred and forty-nine samples, previously unpublished, had received adjuvant tamoxifen only (tam-treated dataset). All samples were required to be ER-positive by protein ligand binding assay.

Microarray analysis was performed with Affymetrix™ U113A Genechips® (Affymetrix, Santa Clara, Calif.). This dataset contained samples from the John Radcliffe Hospital, Oxford, U.K., Guys Hospital, London, U.K. and Uppsala University Hospital, Uppsala, Sweden. Samples from Oxford and London were processed at the Jules Bordet Institute in Brussels, Belgium. For the samples from Uppsala, RNA was extracted at the Karolinska Institute and hybridized at the Genome Institute of Singapore in Singapore. The quality of the RNA obtained from each tumour sample was assessed via the RNA profile generated by the Agilent bioanalyzer. RNA extraction, amplification, hybridization, and scanning were done according to standard Affymetrix protocols. Gene expression values from the CEL were normalized by use of RMA. Each population was normalised separately. Each hospital's institutional ethics board approved the use of the tissue material and written informed consent was obtained. The raw data for the tam-treated dataset are available at the Gene Expression Omnibus repository database (http://www.ncbi.nlm.nih.gov/geo/), with accession code GSE XXX.

The inventors also used four other publicly available datasets, described in recent publications: van de Vijver (n=295), Wang (n=286), Sotiriou (n=99), Sorlie (n=78), in the analysis. For the survival analysis, we used tumors classified as ER-positive only (van de Vijver (n=122), Wang (n=209)). For the survival analysis involving patients who had received no systemic adjuvant treatment, patients from the van de Vijver et al., Wang et al. and previously published dataset were combined (n=417 ER-positive patients, hereby referred to as the “untreated” dataset).

### Data Analysis

### Estrogen (ER) and Progesterone Receptor (PgR) Level

Patients were initially selected at their institutions according to a positive ER status which was determined by protein ligand-binding assay. The inventors subsequently confirmed a positive ER level by using the microarray data. The ER level was measured by probe set (a 30-mer oligonucleotide) on our human Affymetrix™ GeneChip® U133 A&B microarray. The inventors have used the probe set “205225_at” for ER. PgR was represented by the probe set “208305_at”. The immunohistochemical measurement of ER is known to correlate with mRNA levels of ER. Tumours with any positive expression level of ER and PgR were considered.

### Histological Grade

Histological grade was based on the Elston-Ellis grading system. A central pathologist reviewed the histological grade and ER status for all samples from Uppsala, Sweden, Guys Hospital, London, UK and the Van de Vijver et al. dataset.

### An Index Based on the Expression of Proliferation-Related Genes to Quantify Genomic Grade: Gene Expression Grade Index (GGI)

“Gene expression grade index” (GGI) is a linear combination of the expression of 128 probe sets (97 genes) that were found to be differentially expressed between histological grade 1 and 3 (see definitions). The index is effectively, a quantification of the degree of similarity between the tumour expression profile and tumour grade. A high gene-expression grade index corresponds to a high grade and vice versa. This index was used to divide each data set into high and low grade sub-groups.

Mapping of probes between microarray platforms was done through Unigene (build #180), according to the method in Praz et al.

### Hierarchical Clustering

The “Cluster” program was used to perform average linkage hierarchical cluster analysis²⁸ after median centering of each gene using an uncentered Pearson correlation as similarity measurement. The cluster results were viewed using “TreeView”. Expression data was downloaded and extracted from datasets Sorlie et al. and Sotiriou et al. The samples were ordered according to subtype as in the original publications to investigate the relation between the expression of the genes in the GGI and the subtypes.

### Statistical Analysis

In order to assess the relation between survival and some continuous variable, a variant of a method introduced to compute the expected survival for individual was used: “Rate of distant recurrence” plots (ref: Terry M. Therneau and Patricia M. grambsch, 2000, “Modeling Survival Data: Extending the Cox Model”, chapter 10). The expected proportion of distant metastasis with respect to the GGI, ER and PgR was plotted using a Cox model fitted with only the variable under study.

Survival curves were visualized using Kaplan-Meier plots and compared using log-rank tests. The univariate and multivariate hazard ratios (HR) were estimated using Cox regression analysis. All statistical tests were two-sided. Statistical analysis was performed using SPSS statistical software package, version 11.5.

### Results

### Applying Genomic Grade to the Previously Reported Molecular Subtypes

To investigate the expression of the gene expression grade index (GGI) in relation to the subtypes, expression data were extracted from data sets Sorlie and Sotiriou et al., the original and confirmatory publications respectively. The genes were clustered using average-linkage clustering and the samples were ordered according to the subtypes as presented in these published manuscripts. Applying genomic grade to the previously reported molecular subtypes (6a: Sorlie et al.; 6b: Sotiriou et al.) Subtypes are ordered the same as in the original publications. The heatmap of GGI genes is placed below the dendrogram. Boxplots of the GGI score (median and range) are placed below each subtype. High grade is indicated by a GGI score >1 and vice versa.

FIG. 6 shows the results of this analysis. In general, the ER-negative subtypes, the basal and the erbB2 subtypes, had high expression of GGI, or were of high grade. However, the ER-positive subtypes showed a diverse range of GGI levels, particularly the luminal C or 3 subtype both highly expressing these proliferation-associated genes, whereas luminal A or 1, and the normal-like were mostly negative for the expression of the GGI, or low grade. This confirmed the hypothesis that there are varying degrees of contribution of cell cycle genes to the biological makeup of ER-positive tumours, whereas ER-negative tumours seem to consistently have over-expression of these genes. It is interesting to note the similarity in expression profiles of the GGI genes between the high grade ER-positive subtype and the ER-negative subtypes.

### Clinical Relevance of ER-Positive Luminal Subtypes as Defined by Genomic Grade

Genomic grade could distinguish clinically subtypes within the ER-positive tumours and the prognostic value of these genomic grade defined subtypes were an improvement over current traditional methods, such as that based on quantitative levels of estrogen and progesterone receptor levels. A Kaplan-Meier survival analysis was performed comparing classes of ER-positive tumours according to GGI score (high vs. low grade) and expression levels of estrogen and progesterone receptor (rich vs. poor expression) with respect to time to distant metastasis (TDM), which is often used as a surrogate for breast cancer specific survival (FIG. 7-KM and Cox). Kaplan Meier survival curves for distant metastasis free survival for GGI (high vs. low), ER expression levels and PgR expression levels (rich vs. poor). FIG. 7a displays the results for the untreated dataset (n=417). FIG. 7b for the tamoxifen-treated dataset (n=249). For the untreated dataset, results shown were combined from multiple datasets involving 417 ER-positive samples hybridized using two popular commercially available oligonucleotide microarray platforms-Affymetrix™ and Agilent™ (see methods). As shown, for both untreated and tamoxifen-treated populations, the expression levels of the ER did not have any prognostic value (p=0.74 and 0.51 respectively). In contrast, both the GGI and expression levels of the PgR had prognostic value (untreated: p<0.0001 for both GGI and PgR; tam-treated: GGI p<0.0001, PgR p=0.0058). The luminal low grade subtype had a much better 10-year estimate of TDM compared with the luminal high grade subtype.

Table 4 shows the univariate and multivariate analysis with other standard prognostic covariates of age, grade, tumour size as well as genomic grade. In the multivariate Cox regression analysis, only the GGI retained significant prognostic value (untreated: HR 2.3 (95% CI: 1.2-4.3; p=0.008; tam-treated: HR 2.14 (95% CI: 1.04-4.02; p=0.0038), subsuming those factors that were significant at the univariate level, including the progesterone receptor expression levels (p=0.3). For the untreated population, tumour size also retained significance in the multivariate model (HR 2.2 (95% CI:1.2-3.8, p=0.0068). This suggests that genomic grade, as measured by the GGI, can distinguish clinically distinct groups of patients within those that express positive levels of estrogen receptor. Furthermore, the GGI had highly significant prognostic value, suggesting a better ability to discriminate clinical outcome over these traditional factors. The ER-positive high grade subgroup's worse disease outcome in the tamoxifen-treated dataset seems to suggest that adjuvant tamoxifen does not alter this subtype's natural disease history despite having a positive ER status. This could potentially flag a group of tumours worthy of further investigation from both a biological and therapeutic standpoint.

As further demonstration of the GGI's prognostic value in ER-positive tumours, the inventors generated figures displaying the rate of distant recurrence as continuous function of the GGI and compared this to continuous levels of ER and PgR for both untreated and tam-treated populations.

Two subtypes of tumours can be distinguished within patients whose breast cancers express at least some level of estrogen receptor. In patients whose tumours express a high level of the genes that comprise the GGI, i.e. corresponding to high genomic grade, their disease outcome was clearly different, with a higher incidence of relapses compared with tumours of low genomic grade. Furthermore, their worse disease outcome seemed unchanged even when given adjuvant tamoxifen, suggesting that this group of women do not seem to benefit from adjuvant tamoxifen despite their positive estrogen receptor values. Note that none of the patients in this study had received adjuvant chemotherapy, so it is unclear if chemotherapy can alter this group's natural disease history. The potential clinical significance of this finding is also underscored by the similarities between the high grade ER-positive group and the high grade ER-negative tumours (basal and erbB2), further suggesting that high levels of expression of the genes associated with high genomic grade is associated with a poor prognosis. The GGI can consistently identify these two groups across multiple datasets which were hybridized using several micro-array platforms, involving 666 ER-positive samples, suggesting our conclusions are robust and highly reproducible than that produced previously by hierarchical cluster analysis.

The genes present in the GGI are associated with cell cycle progression and proliferation: among the top 20 overexpressed genes were UBE2C, KPNA2, TPX2, FOXM1, STK6, CCNA2, BIRC5, and MYBL2). For ER-positive tumours, genomic grade was associated with differing relapse-free survival, but for ER-negative tumours, as almost all are associated with high genomic grade, the GGI had no prognostic value. Therefore, cell-cycle related genes seem to have prognostic value only in breast cancer patients with positive expression of ER. Within this group, the incidence of distant metastases seems to be predominantly driven by this set of proliferation and grade-derived genes. However, in ER-negative tumours, there may be further factors driving the underlying biology of metastasis besides cell-cycle associated genes. The prognostic ability of a “cell proliferation signature” in a subset of patients has been reported previously in women who express relatively high estrogen receptor expression for their age⁵. The analysis of the ER-positive subgroups was divided by genomic grade to the previously described luminal subgroups and this concept was validated in over 650 patients. Furthermore, genomic grade remains the strongest variable in univariate and multivariate analysis (Table 4 or 5) that takes clinical prognostic factors into consideration.

Currently there are several molecular signatures derived from microarray technology that claim to be able to predict prognosis in breast cancer patients. Some of these gene signatures reported can predict clinical outcome in ER-positive tumours treated with adjuvant tamoxifen. In the recurrence score developed by Paik et al. the proliferation set of five genes had the largest hazard ratios in their large training and validation sets and the highest “weight” or coefficient in their recurrence score formula indicating their high importance in deriving a prognosis classification for women with early stage breast cancer treated with adjuvant tamoxifen. Proliferation-related genes appear to be an important—if not the most important—component of many existing prognostic gene signatures for breast cancer that are based on gene-expression profiles. By using the 11 genes in common between the GGI and a 70-gene prognostic gene classifier for women with early stage breast cancer under the age of 55, similar survival curves to the validation publication were obtained, suggesting that grade-related genes constitute a significant amount of the prognostic power of this signature. The subgroups achieved by these prognostic signatures and that obtained by the classification of ER-positive tumours by genomic grade overlap significantly because of a strong dependence on cell-cycle genes to drive metastasis and relapse. The advantage of this approach is that the biological mechanism that is responsible for the poor outcome is obvious, rather than a gene set that likely represents a variety of molecular functions and biological processes. Because antiestrogens such as tamoxifen have a cell cycle-specific action on breast cancer cells and influence the expression and activity of several cell cycle-regulatory molecules, the development of aberrant cell cycle control mechanisms is an obvious mechanism by which cells might develop resistance to antiestrogens. It is currently incompletely understood why up to 30-40% of ER-positive breast cancers develop resistance to tamoxifen when positive expression of the ER is the best predictor predictors of tamoxifen response in the clinical setting. Over-expression of cyclin D1, a critical controller of the cell cycle, has been associated with tamoxifen resistance and can reverse the growth-inhibitory effect of antiestrogens in estrogen receptor-positive breast cancer cells. Further investigation into the oncogenic pathways that drive the cell cycle machinery will be beneficial in developing new agents to treat the high grade subgroup.

Definition of clinically relevant tumour subclasses within ER-positive breast cancers is of great importance to the treating oncologist today. The emergence of new strategies of adjuvant anti-estrogen therapy as well as new chemotherapeutic and biological agents has made treatment decision making for women with early stage breast cancer sometimes a difficult task. Previously, tamoxifen was the mainstay of anti-estrogen therapy, with significant reductions in the risk or relapse, death and contralateral breast cancer for women with early stage, ER-positive breast cancer³⁸. However, since the advent of aromatase inhibitors and the reporting of several trials finding them to be more effective than tamoxifen in postmenopausal women, the American Society of Clinical Oncology has recommended that an aromatase inhibitor be included in the therapy of postmenopausal women with early stage hormone responsive breast cancers. However, it is still unclear the best combination and sequencing of aromatase inhibitors and tamoxifen, and whether all women with ER-positive tumours derive the same or differing benefit from these agents. The elucidation of clinically relevant and biological distinct hormone responsive breast tumour phenotypes can help facilitate the optimization of such therapy as they may require different therapeutic strategies.

In conclusion, the use of genomic grade can distinguish two subtypes with ER-positive breast cancers in a reproducible manner across multiple datasets and micro-array platforms. This is validated ept in over 650 ER-positive breast cancer samples. These subgroups have statistically distinct clinical outcome in both systemically untreated and tamoxifen-only treated populations. Stratification by subtype in clinical trials may provide important information on the potentially diverse effect of endocrine therapies, chemotherapies and biological agents on these subgroups. A focussed biological investigation into these distinct phenotypes may result in identification of separate and different therapeutic targets.

The genes identified herein may be used to generate a model capable of predicting the breast cancer grade of an unknown breast cell sample based on the expression of the identified genes in the sample. Such a model may be generated by any of the algorithms described herein or otherwise known in the art as well as those recognized as equivalent in the art using gene(s) (and subsets thereof) disclosed herein for the identification of whether an unknown or suspicious breast cancer sample is normal or is in one or more stages and/or grades of breast cancer. The model provides a means for comparing expression profiles of gene(s) of the subset from the sample against the profiles of reference data used to build the model. The model can compare the sample profile against each of the reference profiles or against model defining delineations made based upon the reference profiles. Additionally, relative values from the sample profile may be used in comparison with the model or reference profiles.

In a preferred embodiment of the invention, breast cell samples identified as normal and non-normal and/or atypical from the same subject may be analyzed for their expression profiles of the genes used to generate the model. This provides an advantageous means of identifying the stage of the abnormal sample based on relative differences from the expression profile of the normal sample. These differences can then be used in comparison to differences between normal and individual abnormal reference data which was also used to generate the model. The detection of gene expression from the samples may be by use of a single micro-array able to assay gene expression. One method of analyzing such data would be from all pairwise comparisons disclosed herein for convenience and accuracy.

Other uses of the present invention include providing the ability to identify breast cancer cell samples as being those of a particular stage and/or grade of cancer for further research or study. This provides a particular advantage in many contexts requiring the identification of breast cancer stage and/or grade based on objective genetic or molecular criteria rather than cytological observation. It is of particular utility to distinguish different grades of a particular breast cancer stage for further study, research or characterization.

The materials for use in the methods of the present invention are ideally suited for preparation of kits produced in accordance with well known procedures. The invention thus provides kits comprising agents for the detection of expression of the disclosed genes for identifying breast cancer stage. Such kits optionally comprise the agent with an identifying description or label or instructions relating to their use in the methods of the present invention, is provided. Such a kit may comprise containers, each with one or more of the various reagents (typically in concentrated form) utilized in the methods, including, for example, pre-fabricated micro-arrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset of the disclosed genes to the exclusion of material irrelevant to the identification of breast cancer stages in a cell containing sample.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

As above described proliferation capture by the Genomic Grade Index (GGI) is an important prognostic factor in breast cancer, for beyond estrogen receptor status and may encompass a significant portion of the predictive power of many previously published prognostic signatures. Inventors were also able to convert and validate by qRT-PCR assay the prognostic value of GGI using frozen (FS) and paraffin-embedded tumor samples (FFPE) from early breast cancer patients. Inventors have developed a qRT-PCR assay based on 8 selected GGI genes involved in different phases of the cell cycle and 4 reference genes. These selected genes are CNB1, CCNA2, CDC2, CDC20, MCM2, MYBL2, KPNA2 and STK6 (4 reference genes are TFRC, GUS, RPLPO and TBP). The preferred 4 selected genes are either CDC2, CDC20, CCNB1 and MCM2 (assay 1) or more preferably CDC2, CDC20, MYBL2 and KPNA2 (assay 2).

The inventors have tested the accuracy of this qRT-PCR assay in concordance with the original micro-array derived GGI above described by using breast cancer population from which frozen, paraffin-embedded tumor samples tissues and micro-array data were available (N=30). A statistically significant correlation was observed between GGI generated by micro-array and qRT-PCR assays (1 and 2) using frozen material (for assay 2: HR=0.945, (95% CI: 0.856-0.98, p=3.67E-09) and FFPE material (for assay 2: HR=0.889, (95% CI:0.721-0.958), p=8.26E-07) as well as between GGI using qRT-PCR derived from frozen and FFPE tumor samples assays (1 and 2) (for assay 2: HR=0.851, 95% CI: 0.636-0.943), p=7.73E-06).

The prognostic value of the qRT-PCR assay 1 and 2 has been tested upon a population of 78 hormono-dependant breast tumor of frozen sample tissue. Statistically significant correlation was observed between a high relapsing risk and an elevated expression of these 4 genes of the bio-assay 1 and 2 (HR for bioassay 2=3.338(95% CI:1.189-9.374), p=0.022). The prognostic value of the bio-assay 1 and 2 remains significative during multivariable analyses (HR for bioassay 2=3.267 (95% CI:1.157-9.227), p=0.025) together with age (<50 years) and tumor size (>2 cm).

The inventors have also assessed the prognostic value of this assay 2 on a population of 208 breast cancers operated consecutively at the Bordet Institute between 1995 and 1996.

These samples are paraffin-embedded tumor sample tissues. Statistically significant correlation has been observed between the high relapsing risk and high expression of the 4 genes of this bio-assay in global population (HR=1.072 (95% CI:0.999-3.507), p=0.050) and in particular in sub-population of breast cancers hormone-dependant (HR=2.26(95% CI:1.075-4.751), p=0.032).

The prognostic value remains significant even during multivariable analyses together with nodal invasion for the global population (HR=1.880(95% CI:0.941-3.757), p=0.074) and the ER positive subgroup (HR=2.249(95% CI:0.982-5.150),p=0.055).

This prognostic value of the bio-assay 2 has been also validated upon another independent population of 106 paraffin-embedded breast tumor sample with similar results.

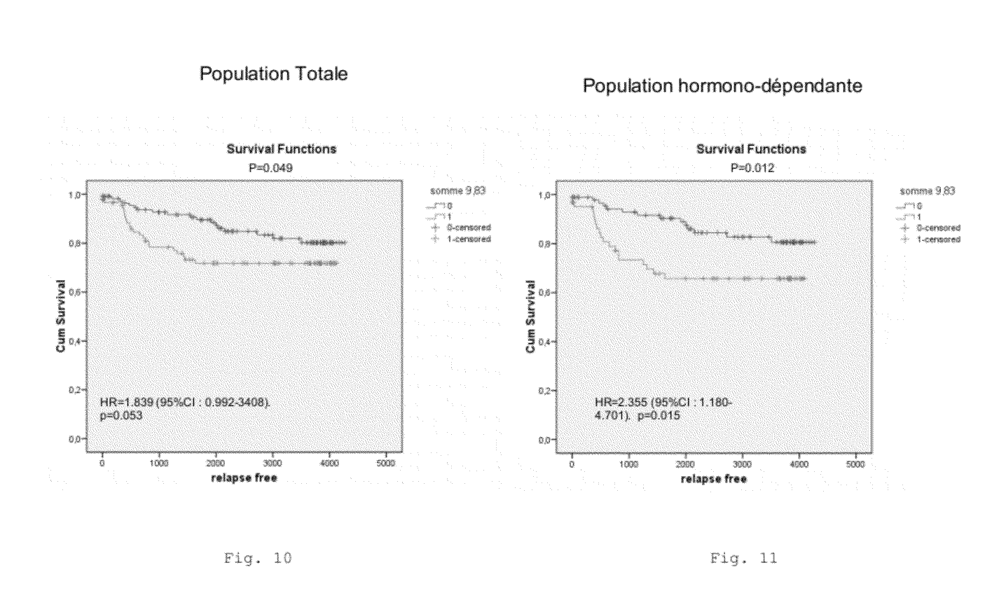

A bio-assay based upon a limited number of genes, such as the four genes selected from the set of genes as described in the present invention, preferably a qRT-PCR assays (assay 1 or assay 2) allows an accurate and reproducible manner the prognostic power of micro-array derived GGI using both frozen and paraffin-embedded tumor samples. As described in the FIGS. 8 to 11 prognostic value of qRT-PCR assay 2 is comparable to a prognostic value of micro-array. This could be applied to patient expressing estrogen receptor.

Different embodiments of the present invention have been described according to the present invention. Many modifications and variations may be made to the techniques and structures described and illustrated herein without departing from the spirit and scope of the invention. Accordingly, it should be understood that the apparatuses described herein are illustrative only and are not limiting upon the scope of the invention.

TABLE 3

| | p-values | | | | | | |
|---|---|---|---|---|---|---|---|
| No. | D | FD > 0 | FD > 1 | FD > 2 | probeset | gene symbol | description |

up-regulated in grade 3 tumors

| | | | | | | | |
|---|---|---|---|---|---|---|---|
| 1 | 2.1162 | 0.0001 | 0.0001 | 0.0001 | 202954 at | UBE2C | ubiquitin-conjugating enzyme E2C |
| 2 | 1.9037 | 0.0001 | 0.0001 | 0.0001 | 222077 s at | RACGAP1 | Rac GTPase activating protein 1 |
| 3 | 1.7292 | 0.0001 | 0.0001 | 0.0001 | 201088 at | KPNA2 | karyopherin alpha 2 (RAG cohort 1, importin alpha |
| | | | | | | | 1) |
| 4 | 1.7264 | 0.0001 | 0.0001 | 0.0001 | 218542 at | C10orf3 | chromosome 10 open reading frame 3 |
| 5 | 1.7259 | 0.0001 | 0.0001 | 0.0001 | 203554 x at | PTTG1 | pituitary tumor-transforming 1 |
| 6 | 1.7053 | 0.0001 | 0.0001 | 0.0001 | 218355 at | KIF4A | kinesin family member 4A |
| 7 | 1.6600 | 0.0001 | 0.0001 | 0.0001 | 210052 s at | TPX2 | TPX2, microtubule-associated protein homolog |
| | | | | | | | (Xenopus laevis) |
| 8 | 1.6598 | 0.0001 | 0.0001 | 0.0001 | 202580 x at | FOXM1 | forkhead box M1 |
| 9 | 1.6548 | 0.0001 | 0.0001 | 0.0001 | 208079 s at | STK6 | serine/threonine kinase 6 |
| 10 | 1.6513 | 0.0001 | 0.0001 | 0.0001 | 204092 s at | STK6 | serine/threonine kinase 6 |
| 11 | 1.6495 | 0.0001 | 0.0001 | 0.0001 | 218755 at | KIF20A | kinesin family member 20A |
| 12 | 1.6387 | 0.0001 | 0.0001 | 0.0001 | 201584 s at | DDX39 | DEAD (Asp-Glu-Ala-Asp) box polypeptide 39 |
| 13 | 1.6347 | 0.0001 | 0.0001 | 0.0001 | 203764 at | DLG7 | discs, large homolog 7 (Drosophila) |
| 14 | 1.6223 | 0.0001 | 0.0001 | 0.0001 | 204825 at | MELK | maternal embryonic leucine zipper kinase |
| 15 | 1.6213 | 0.0001 | 0.0001 | 0.0001 | 203418 at | CCNA2 | cyclin A2 |
| 16 | 1.6095 | 0.0001 | 0.0001 | 0.0001 | 204766 s at | NUDT1 | nudix (nucleoside diphosphate linked moiety X) |
| | | | | | | | type motif 1 |
| 17 | 1.6057 | 0.0001 | 0.0001 | 0.0001 | 206102 at | KIAA0186 | KIAA0186 gene product |
| 18 | 1.5986 | 0.0001 | 0.0001 | 0.0001 | 202095 s at | BIRC5 | baculoviral IAP repeat-containing 5 (survivin) |
| 19 | 1.5957 | 0.0001 | 0.0001 | 0.0001 | 201710 at | MYBL2 | v-myb myeloblastosis viral oncogene homolog |
| | | | | | | | (avian)-like 2 |
| 20 | 1.5879 | 0.0002 | 0.0001 | 0.0001 | 211762 s at | KPNA2 | karyopherin alpha 2 (RAG cohort 1, importin alpha |
| | | | | | | | 1) |
| 21 | 1.5816 | 0.0002 | 0.0001 | 0.0001 | 209680 s at | KIFC1 | Kinesin family member C1 |
| 22 | 1.5785 | 0.0002 | 0.0001 | 0.0001 | 209408 at | KIF2C | kinesin family member 2C |
| 23 | 1.5671 | 0.0002 | 0.0001 | 0.0001 | 219918 s at | ASPM | asp (abnormal spindle)-like, microcephaly |
| | | | | | | | associated (Drosophila) |
| 24 | 1.5650 | 0.0003 | 0.0001 | 0.0001 | 203145 at | SPAG5 | sperm associated antigen 5 |
| 25 | 1.5595 | 0.0003 | 0.0001 | 0.0001 | 204962 s at | CENPA | centromere protein A, 17 kDa |
| 26 | 1.5551 | 0.0003 | 0.0001 | 0.0001 | 202870 s at | CDC20 | CDC20 cell division cycle 20 homolog (S. cerevisiae) |
| 27 | 1.5446 | 0.0003 | 0.0001 | 0.0001 | 38158 at | ESPL1 | extra spindle poles like 1 (S. cerevisiae) |
| 28 | 1.5376 | 0.0003 | 0.0001 | 0.0001 | 202107 s at | MCM2 | MCM2 minichromosome maintenance deficient |
| | | | | | | | 2, mitotin (S. cerevisiae) |
| 29 | 1.5236 | 0.0004 | 0.0001 | 0.0001 | 204767 s at | FEN1 | flap structure-specific endonuclease 1 |
| 30 | 1.5226 | 0.0004 | 0.0001 | 0.0001 | 203046 s at | TIMELESS | timeless homolog (Drosophila) |
| 31 | 1.5221 | 0.0004 | 0.0001 | 0.0001 | 221677 s at | DONSON | downstream neighbor of SON |
| 32 | 1.5134 | 0.0005 | 0.0001 | 0.0001 | 210559 s at | CDC2 | cell division cycle 2, G1 to S and G2 to M |
| 33 | 1.5047 | 0.0006 | 0.0001 | 0.0001 | 221520 s at | CDCA8 | cell division cycle associated 8 |
| 34 | 1.5017 | 0.0007 | 0.0001 | 0.0001 | 214710 s at | CCNB1 | cyclin B1 |
| 35 | 1.4945 | 0.0007 | 0.0001 | 0.0001 | 209714 s at | CDKN3 | cyclin-dependent kinase inhibitor 3 (CDK2 |
| | | | | | | | associated dual specificity phosphatase) |
| 36 | 1.4933 | 0.0008 | 0.0001 | 0.0001 | 204444 at | KIF11 | kinesin family member 11 |
| 37 | 1.4927 | 0.0008 | 0.0001 | 0.0001 | 210821 x at | CENPA | centromere protein A, 17 kDa |
| 38 | 1.4915 | 0.0008 | 0.0001 | 0.0001 | 218726 at | DKFZp762E1312 | hypothetical protein DKFZp762E1312 |
| 39 | 1.4895 | 0.0009 | 0.0001 | 0.0001 | 220651 s at | MCM10 | MCM10 minichromosome maintenance |
| | | | | | | | deficient 10 (S. cerevisiae) |
| 40 | 1.4865 | 0.0010 | 0.0001 | 0.0001 | 201475 x at | MARS | methionine-tRNA synthetase |
| 41 | 1.4715 | 0.0014 | 0.0001 | 0.0001 | 204033 at | TRIP13 | thyroid hormone receptor interactor 13 |
| 42 | 1.4672 | 0.0014 | 0.0001 | 0.0001 | 202705 at | CCNB2 | cyclin B2 |
| 43 | 1.4624 | 0.0014 | 0.0001 | 0.0001 | 204649 at | TROAP | trophinin associated protein (tastin) |
| 44 | 1.4603 | 0.0014 | 0.0001 | 0.0001 | 220060 s at | FLJ20641 | hypothetical protein FLJ20641 |
| 45 | 1.4534 | 0.0016 | 0.0001 | 0.0001 | 209836 x at | LAT1-3TM | LAT1-3TM protein |
| 46 | 1.4533 | 0.0016 | 0.0001 | 0.0001 | 203276 at | LMNB1 | lamin B1 |
| 47 | 1.4471 | 0.0018 | 0.0001 | 0.0001 | 205034 at | CCNE2 | cyclin E2 |
| 48 | 1.4455 | 0.0018 | 0.0001 | 0.0001 | 203213 at | CDC2 | cell division cycle 2, G1 to S and G2 to M |
| 49 | 1.4384 | 0.0019 | 0.0001 | 0.0001 | 209464 at | AURKB | aurora kinase B |
| 50 | 1.4381 | 0.0019 | 0.0001 | 0.0001 | 205046 at | CENPE | centromere protein E, 312 kDa |
| 51 | 1.4373 | 0.0019 | 0.0001 | 0.0001 | 203755 at | BUB1B | BUB1 budding uninhibited by benzimidazoles 1 |
| | | | | | | | homolog beta (yeast) |
| 52 | 1.4336 | 0.0019 | 0.0001 | 0.0001 | 203214 x at | CDC2 | cell division cycle 2, G1 to S and G2 to M |
| 53 | 1.4236 | 0.0022 | 0.0002 | 0.0001 | 214804 at | FSHPRH1 | FSH primary response (LRPR1 homolog, rat) 1 |
| 54 | 1.4167 | 0.0026 | 0.0003 | 0.0001 | 212949 at | BRRN1 | barren homolog (Drosophila) |
| 55 | 1.4134 | 0.0027 | 0.0003 | 0.0001 | 204318 s at | GTSE1 | G-2 and S-phase expressed 1 |
| 56 | 1.4105 | 0.0030 | 0.0003 | 0.0001 | 207165 at | HMMR | hyaluronan-mediated motility receptor |
| | | | | | | | (RHAMM) |
| 57 | 1.4079 | 0.0031 | 0.0003 | 0.0001 | 212022 s at | MKI67 | antigen identified by monoclonal antibody Ki-67 |
| 58 | 1.4051 | 0.0031 | 0.0003 | 0.0001 | 213226 at | CCNA2 | cyclin A2 |
| 59 | 1.3931 | 0.0041 | 0.0004 | 0.0001 | 219510 at | POLQ | polymerase (DNA directed), theta |
| 60 | 1.3890 | 0.0044 | 0.0004 | 0.0001 | 204026 s at | ZWINT | ZW10 interactor |
| 61 | 1.3890 | 0.0044 | 0.0004 | 0.0001 | 203432 at | TMPO | thymopoietin |
| 62 | 1.3872 | 0.0046 | 0.0004 | 0.0001 | 204768 s at | FEN1 | flap structure-specific endonuclease 1 |
| 63 | 1.3855 | 0.0047 | 0.0004 | 0.0001 | 209773 s at | RRM2 | ribonucleotide reductase M2 polypeptide |
| 64 | 1.3847 | 0.0047 | 0.0004 | 0.0001 | 214431 at | GMPS | guanine monphosphate synthetase |
| 65 | 1.3842 | 0.0048 | 0.0004 | 0.0001 | 212023 s at | MKI67 | antigen identified by monoclonal antibody Ki-67 |
| 66 | 1.3752 | 0.0052 | 0.0004 | 0.0002 | 218883 s at | MLF1IP | MLF1 interacting protein |
| 67 | 1.3541 | 0.0077 | 0.0006 | 0.0003 | 211519 s at | KIF2C | kinesin family member 2C |
| 68 | 1.3503 | 0.0083 | 0.0006 | 0.0003 | 202240 at | PLK1 | polo-like kinase 1 (Drosophila) |
| 69 | 1.3460 | 0.0089 | 0.0007 | 0.0003 | 205733 at | BLM | Bloom syndrome |
| 70 | 1.3457 | 0.0092 | 0.0008 | 0.0003 | 222039 at | LOC146909 | hypothetical protein LOC146909 |
| 71 | 1.3443 | 0.0096 | 0.0008 | 0.0003 | 209642 at | BUB1 | BUB1 budding uninhibited by benzimidazoles 1 |
| | | | | | | | homolog (yeast) |
| 72 | 1.3376 | 0.0102 | 0.0010 | 0.0003 | 213599 at | OIP5 | Opa-interacting protein 5 |
| 73 | 1.3372 | 0.0102 | 0.0010 | 0.0003 | 214096 s at | SHMT2 | serine hydroxymethyltransferase 2 (mitochondrial) |
| 74 | 1.3348 | 0.0105 | 0.0012 | 0.0003 | 211072 x at | K-ALPHA-1 | tubulin, alpha, ubiquitous |
| 75 | 1.3237 | 0.0130 | 0.0017 | 0.0004 | 202779 s at | UBE2S | ubiquitin-conjugating enzyme E2S |
| 76 | 1.3226 | 0.0133 | 0.0017 | 0.0004 | 218447 at | DC13 | DC13 protein |
| 77 | 1.3215 | 0.0138 | 0.0017 | 0.0004 | 213911 s at | H2AFZ | H2A histone family, member Z |
| 78 | 1.3211 | 0.0138 | 0.0017 | 0.0004 | 212141 at | MCM4 | MCM4 minichromosome maintenance deficient 4 |
| | | | | | | | (S. cerevisiae) |
| 79 | 1.3156 | 0.0153 | 0.0019 | 0.0005 | 221591 s at | FLJ10156 | hypothetical protein FLJ10156 |
| 80 | 1.3139 | 0.0162 | 0.0019 | 0.0005 | 204822 at | TTK | TTK protein kinase |
| 81 | 1.3121 | 0.0165 | 0.0020 | 0.0005 | 209251 x at | TUBA6 | tubulin alpha 6 |
| 82 | 1.3086 | 0.0173 | 0.0023 | 0.0006 | 217835 x at | C20or124 | chromosome 20 open reading frame 24 |
| 83 | 1.3081 | 0.0176 | 0.0023 | 0.0006 | 201890 at | RRM2 | ribonucleotide reductase M2 polypeptide |
| 84 | 1.3059 | 0.0184 | 0.0024 | 0.0006 | 213671 s at | MARS | methionine-tRNA synthetase |
| 85 | 1.3053 | 0.0185 | 0.0024 | 0.0006 | 218009 s at | PRC1 | protein regulator of cytokinesis 1 |
| 86 | 1.3010 | 0.0197 | 0.0026 | 0.0007 | 207828 s at | CENPF | centromere protein F, 350/400ka (mitosin) |
| 87 | 1.3002 | 0.0198 | 0.0026 | 0.0007 | 219555 s at | BM039 | uncharacterized bone marrow protein BM039 |
| 88 | 1.2969 | 0.0206 | 0.0026 | 0.0007 | 204695 at | CDC25A | cell division cycle 25A |
| 89 | 1.2953 | 0.0214 | 0.0026 | 0.0009 | 212021 s at | MKI67 | antigen identified by monoclonal antibody Ki-67 |
| 90 | 1.2898 | 0.0229 | 0.0028 | 0.0009 | 201090 x at | K-ALPHA-1 | tubulin, alpha, ubiquitous |
| 91 | 1.2885 | 0.0233 | 0.0033 | 0.0010 | 218039 at | NUSAP1 | nucleolar and spindle associated protein 1 |
| 92 | 1.2851 | 0.0246 | 0.0034 | 0.0012 | 204603 at | EXO1 | exonuclease 1 |
| 93 | 1.2846 | 0.0248 | 0.0034 | 0.0012 | 203362 s at | MAD2L1 | MAD2 mitotic arrest deficient-like 1 (yeast) |
| 94 | 1.2845 | 0.0248 | 0.0034 | 0.0012 | 202094 at | BIRC5 | baculoviral IAP repeat-containing 5 (survivin) |
| 95 | 1.2840 | 0.0249 | 0.0034 | 0.0012 | 204162 at | KNTC2 | kinetochore associated 2 |
| 96 | 1.2825 | 0.0254 | 0.0037 | 0.0012 | 222036 s at | MCM4 | MCM4 minichromosome maintenance deficient 4 |
| | | | | | | | (S. cerevisiae) |
| 97 | 1.2780 | 0.0272 | 0.0039 | 0.0014 | 204252 at | CDK2 | cyclin-dependent kinase 2 |
| 98 | 1.2775 | 0.0274 | 0.0039 | 0.0014 | 219000 s at | DCC1 | defective in sister chromatid cohesion homolog 1 |
| | | | | | | | (S. cerevisiae) |
| 99 | 1.2772 | 0.0277 | 0.0041 | 0.0014 | 201524 x at | UBE2N | ubiquitin-conjugating enzyme E2N (UBC13 homolog, |
| | | | | | | | yeast) |
| 100 | 1.2694 | 0.0294 | 0.0044 | 0.0018 | 204817 at | ESPL1 | extra spindle poles like 1 (S. cerevisiae) |
| 101 | 1.2657 | 0.0313 | 0.0046 | 0.0019 | 218662 s at | HCAP-G | chromosome condensation protein G |
| 102 | 1.2620 | 0.0324 | 0.0052 | 0.0022 | 206364 at | KIF14 | Kinesin family member 14 |
| 103 | 1.2612 | 0.0329 | 0.0054 | 0.0022 | 221436 s at | CDCA3 | cell division cycle associated 3 |
| 104 | 1.2609 | 0.0331 | 0.0054 | 0.0022 | 201195 s at | SLC7A5 | solute carrier family 7 (cationic amino acid transporter, |
| | | | | | | | y+ system), member 5 |
| 105 | 1.2602 | 0.0332 | 0.0055 | 0.0022 | 208696 at | CCT5 | chaperonin containing TCP1, subunit 5 (epsilon) |
| 106 | 1.2553 | 0.0358 | 0.0059 | 0.0023 | 218556 at | ORMDL2 | ORM1-like 2 (S. cerevisiae) |
| 107 | 1.2476 | 0.0400 | 0.0069 | 0.0024 | 211058 x at | K-ALPHA-1 | tubulin, alpha, ubiquitous |
| 108 | 1.2458 | 0.0407 | 0.0072 | 0.0024 | 212723 at | PTDSR | phosphatidylserine receptor |
| 109 | 1.2447 | 0.0414 | 0.0076 | 0.0024 | 203022 at | RNASEH2A | ribonuclease H2, large subunit |
| 110 | 1.2402 | 0.0450 | 0.0084 | 0.0029 | 210334 x at | BIRC5 | baculoviral IAP repeat-containing 5 (survivin) |
| 111 | 1.2383 | 0.0461 | 0.0090 | 0.0031 | 203744 at | HMGB3 | high-mobility group box 3 |
| 112 | 1.2360 | 0.0478 | 0.0095 | 0.0034 | 219306 at | KNSL7 | kinesin-like 7 |
| 113 | 1.2332 | 0.0509 | 0.0102 | 0.0038 | 220865 s at | TPRT | trans-prenyltransferase |
| 114 | 1.2331 | 0.0510 | 0.0103 | 0.0038 | 204641 at | NEK2 | NIMA (never in mitosis gene a)-related kinase 2 |
| 115 | 1.2319 | 0.0518 | 0.0104 | 0.0038 | 203358 s at | EZH2 | enhancer of zeste homolog 2 (Drosophila) |
| 116 | 1.2249 | 0.0581 | 0.0119 | 0.0044 | 213088 s at | DNAJC9 | DnaJ (Hsp40) homolog, subfamily C, member 9 |
| 117 | 1.2233 | 0.0593 | 0.0123 | 0.0047 | 214516 at | HIST1H4B | histone 1, H4b |
| 118 | 1.2226 | 0.0602 | 0.0124 | 0.0049 | 202110 at | COX7B | cytochrome c oxidase subunit VIIb |
| 119 | 1.2214 | 0.0610 | 0.0128 | 0.0050 | 218982 s at | MRPS17 | mitochondrial ribosomal protein S17 |
| 120 | 1.2207 | 0.0615 | 0.0129 | 0.0051 | 205339 at | SIL | TAL1 (SCL) interrupting locus |
| 121 | 1.2206 | 0.0615 | 0.0129 | 0.0051 | 201342 at | SNRPC | small nuclear ribonucleoprotein polypeptide C |
| 122 | 1.2199 | 0.0619 | 0.0131 | 0.0051 | 201678 s at | DC12 | DC12 protein |
| 123 | 1.2192 | 0.0622 | 0.0132 | 0.0052 | 218875 s at | FBXO5 | F-box protein 5 |
| 124 | 1.2160 | 0.0650 | 0.0139 | 0.0055 | 218663 at | HCAP-G | chromosome condensation protein G |
| 125 | 1.2155 | 0.0652 | 0.0141 | 0.0055 | 212020 s at | MKI67 | antigen identified by monoclonal antibody Ki-67 |
| 126 | 1.2065 | 0.0738 | 0.0163 | 0.0065 | 217755 at | HN1 | hematological and neurological expressed 1 |
| 127 | 1.2028 | 0.0777 | 0.0176 | 0.0067 | 202635 s at | POLR2K | polymerase (RNA) II (DNA directed) polypeptide |
| | | | | | | | K, 7.0 kDa |
| 128 | 1.2003 | 0.0802 | 0.0183 | 0.0069 | 202397 at | NUTF2 | nuclear transport factor 2 |
| 129 | 1.1993 | 0.0811 | 0.0183 | 0.0071 | 201930 at | MCM6 | MCM6 minichromosome maintenance deficient 6 |
| | | | | | | | (MIS5 homolog, S. pombe) (S. cerevisiae) |
| 130 | 1.1960 | 0.0849 | 0.0194 | 0.0073 | 222037 at | MCM4 | MCM4 minichromosome maintenance deficient 4 |
| | | | | | | | (S. cerevisiae) |
| 131 | 1.1914 | 0.0900 | 0.0210 | 0.0083 | 205024 s at | RAD51 | RAD51 homolog (RecA homolog, E. coli) (S. cerevisiae) |
| 132 | 1.1884 | 0.0932 | 0.0217 | 0.0090 | 211750 x at | TUBA6 | tubulin alpha 6 |
| 133 | 1.1880 | 0.0938 | 0.0221 | 0.0092 | 203856 at | VRK1 | vaccinia related kinase 1 |
| 134 | 1.1861 | 0.0961 | 0.0229 | 0.0093 | 204267 x at | PKMYT1 | membrane-associated tyrosine-and threonine- |
| | | | | | | | specific cdc2-inhibitory kinase |
| 135 | 1.1807 | 0.1036 | 0.0256 | 0.0107 | 219787 s at | ECT2 | epithelial cell transforming sequence 2 oncogene |
| 136 | 1.1800 | 0.1045 | 0.0258 | 0.0109 | 219494 at | RAD54B | RAD54 homolog B (S. cerevisiae) |
| 137 | 1.1790 | 0.1050 | 0.0262 | 0.0110 | 219990 at | FLJ23311 | FLJ23311 protein |
| 138 | 1.1770 | 0.1078 | 0.0268 | 0.0114 | 219061 s at | DXS9879E | DNA segment on chromosome X (unique) 9879 |
| | | | | | | | expressed sequence |
| 139 | 1.1767 | 0.1083 | 0.0269 | 0.0114 | 203832 at | SNRPF | small nuclear ribonucleoprotein polypeptide F |
| 140 | 1.1757 | 0.1094 | 0.0274 | 0.0115 | 213646 x at | K-ALPHA-1 | tubulin, alpha, ubiquitous |
| 141 | 1.1749 | 0.1107 | 0.0277 | 0.0117 | 201519 at | TOMM70A | translocase of outer mitochondrial membrane 70 |
| | | | | | | | homolog A (yeast) |
| 142 | 1.1728 | 0.1131 | 0.0287 | 0.0121 | 202824 s at | TCEB1 | transcription elongation factor B (SIII), polypeptide |
| | | | | | | | 1 (15 kDa, elongin C) |
| 143 | 1.1725 | 0.1134 | 0.0290 | 0.0122 | 222029 x at | HKE2 | HLA class II region expressed gene KE2 |
| 144 | 1.1714 | 0.1142 | 0.0296 | 0.0127 | 205644 s at | SNRPG | small nuclear ribonucleoprotein polypeptide G |
| 145 | 1.1664 | 0.1222 | 0.0317 | 0.0146 | 204170 s at | CKS2 | CDC28 protein kinase regulatory subunit 2 |
| 146 | 1.1658 | 0.1228 | 0.0321 | 0.0147 | 205394 at | CHEK1 | CHK1 checkpoint homolog (S. pombe) |
| 147 | 1.1630 | 0.1270 | 0.0340 | 0.0155 | 204023 at | RFC4 | replication factor C (activator 1) 4, 37 kDa |
| 148 | 1.1619 | 0.1289 | 0.0345 | 0.0155 | 218151 x at | GPR172A | G protein-coupled receptor 172A |
| 149 | 1.1616 | 0.1290 | 0.0345 | 0.0155 | 202352 s at | PSMD12 | proteasome (prosome, macropain) 26S subunit, |
| | | | | | | | non-ATPase, 12 |
| 150 | 1.1597 | 0.1322 | 0.0362 | 0.0158 | 202188 at | NUP93 | nucleoporin 93 kDa |
| 151 | 1.1548 | 0.1420 | 0.0390 | 0.0175 | 201291 s at | TOP2A | topoisomerase (DNA) II alpha 170 kDa |
| 152 | 1.1528 | 0.1459 | 0.0404 | 0.0179 | 219978 s at | NUSAP1 | nucleolar and spindle associated protein 1 |
| 153 | 1.1525 | 0.1462 | 0.0405 | 0.0182 | 201266 at | TXNRD1 | thioredoxin reductase 1 |
| 154 | 1.1514 | 0.1487 | 0.0415 | 0.0186 | 204126 s at | CDC45L | CDC45 cell division cycle 45-like (S. cerevisiae) |
| 155 | 1.1508 | 0.1497 | 0.0418 | 0.0189 | 209709 s at | HMMR | hyaluronan-mediated motility receptor |
| | | | | | | | (RHAMM) |
| 156 | 1.1501 | 0.1513 | 0.0421 | 0.0189 | 219512 at | C20orf172 | chromosome 20 open reading frame 172 |
| 157 | 1.1466 | 0.1583 | 0.0446 | 0.0204 | 218408 at | TIMM10 | translocase of inner mitochondrial membrane 10 |
| | | | | | | | homolog (yeast) |
| 158 | 1.1444 | 0.1616 | 0.0457 | 0.0215 | 201555 at | MCM3 | MCM3 minichromosome maintenance deficient 3 |
| | | | | | | | (S. cerevisiae) |
| 159 | 1.1413 | 0.1670 | 0.0479 | 0.0223 | 218239 s at | GTPBP4 | GTP binding protein 4 |
| 160 | 1.1412 | 0.1674 | 0.0479 | 0.0223 | 200783 s at | STMN1 | stathmin 1/oncoprotein 18 |
| 161 | 1.1389 | 0.1729 | 0.0498 | 0.0228 | 214095 at | SHMT2 | serine hydroxymethyltransferase 2 (mitochondrial) |
| 162 | 1.1385 | 0.1735 | 0.0503 | 0.0231 | 200853 at | H2AFZ | H2A histone family, member Z |
| 163 | 1.1346 | 0.1818 | 0.0545 | 0.0248 | 203931 s at | MRPL12 | mitochondrial ribosomal protein L12 |
| 164 | 1.1332 | 0.1840 | 0.0554 | 0.0254 | 209744 x at | ITCH | itchy homolog E3 ubiquitin protein ligase (mouse) |
| 165 | 1.1329 | 0.1846 | 0.0560 | 0.0256 | 212639 x at | TUBA3 | tubulin, alpha 3 |
| 166 | 1.1316 | 0.1873 | 0.0576 | 0.0259 | 204044 at | QPRT | quinolinate phosphoribosyltransferase |
| | | | | | | | (nicotinate-nucleotide pyrophosphorylase (carboxylating)) |
| 167 | 1.1254 | 0.2017 | 0.0638 | 0.0296 | 208864 s at | TXN | thioredoxin |
| 168 | 1.1233 | 0.2063 | 0.0661 | 0.0304 | 201114 x at | PSMA7 | proteasome (prosome, macropain) subunit, alpha |
| | | | | | | | type, 7 |
| 169 | 1.1228 | 0.2073 | 0.0666 | 0.0311 | 209172 s at | CENPF | centromere protein F, 350/400ka (mitosin) |
| 170 | 1.1224 | 0.2080 | 0.0672 | 0.0314 | 201577 at | NME1 | non-metastatic cells 1, protein (NM23A) expressed |
| | | | | | | | in |
| 171 | 1.1204 | 0.2129 | 0.0699 | 0.0324 | 213330 s at | STIP1 | stress-induced-phosphoprotein 1 (Hsp70/Hsp90 |
| | | | | | | | organizing protein) |
| 172 | 1.1197 | 0.2142 | 0.0701 | 0.0331 | 218238 at | GTPBP4 | GTP binding protein 4 |
| 173 | 1.1192 | 0.2155 | 0.0708 | 0.0335 | 214437 s at | SHMT2 | serine hydroxymethyltransferase 2 (mitochondrial) |
| 174 | 1.1181 | 0.2180 | 0.0726 | 0.0343 | 218027 at | MRPL15 | mitochondrial ribosomal protein L15 |
| 175 | 1.1178 | 0.2193 | 0.0728 | 0.0346 | 203612 at | BYSL | bystin-like |
| 176 | 1.1173 | 0.2209 | 0.0733 | 0.0347 | 202487 s at | H2AFV | H2A histone family, member V |
| 177 | 1.1099 | 0.2410 | 0.0815 | 0.0399 | 218308 at | TACC3 | transforming, acidic coiled-coil containing protein 3 |
| 178 | 1.1089 | 0.2449 | 0.0823 | 0.0408 | 208511 at | PTTG3 | pituitary tumor-transforming 3 |
| 179 | 1.1070 | 0.2509 | 0.0849 | 0.0421 | 212160 at | XPOT | exportin, tRNA (nuclear export receptor for tRNAs) |
| 180 | 1.1061 | 0.2541 | 0.0863 | 0.0429 | 2028 s at | E2F1 | E2F transcription factor 1 |
| 181 | 1.1037 | 0.2608 | 0.0900 | 0.0449 | 203746 s at | HCCS | holocytochrome c synthase (cytochrome c hemelyase) |
| 182 | 1.1018 | 0.2655 | 0.0934 | 0.0464 | 219004 s at | C21orf45 | chromosome 21 open reading frame 45 |
| 183 | 1.1010 | 0.2681 | 0.0940 | 0.0473 | 206632 s at | APOBEC3B | apolipoprotein B mRNA editing enzyme, catalytic |
| | | | | | | | polypeptide-like 3B |
| 184 | 1.1006 | 0.2691 | 0.0946 | 0.0478 | 219588 s at | MTB | more than blood homolog |
| 185 | 1.1000 | 0.2706 | 0.0953 | 0.0483 | 205393 s at | CHEK1 | CHK1 checkpoint homolog (S. pombe) |

up-regulated in grade 1 tumors

| | | | | | | | |
|---|---|---|---|---|---|---|---|
| 1 | −1.4739 | 0.0014 | 0.0001 | 0.0001 | 213103 at | STARD13 | START domain containing 13 |
| 2 | −1.4647 | 0.0014 | 0.0001 | 0.0001 | 204703 at | TTC10 | tetratricopeptide repeat domain 10 |
| 3 | −1.4196 | 0.0024 | 0.0002 | 0.0001 | 218346 s at | SESN1 | sestrin 1 |
| 4 | −1.4084 | 0.0031 | 0.0003 | 0.0001 | 218471 s at | BBS1 | Bardet-Biedl syndrome 1 |
| 5 | −1.3840 | 0.0048 | 0.0004 | 0.0001 | 205898 at | CX3CR1 | chemokine (C-X3-C motif) receptor 1 |
| 6 | −1.3482 | 0.0084 | 0.0007 | 0.0003 | 204072 s at | 13CDNA73 | hypothetical protein CG003 |
| 7 | −1.3235 | 0.0131 | 0.0017 | 0.0004 | 219455 at | FLJ21062 | hypothetical protein FLJ21062 |
| 8 | −1.2840 | 0.0249 | 0.0034 | 0.0012 | 217889 s at | CYBRD1 | cytochrome b reductase 1 |
| 9 | −1.2835 | 0.0252 | 0.0034 | 0.0012 | 219238 at | FLJ20477 | hypothetical protein FLJ20477 |
| 10 | −1.2663 | 0.0312 | 0.0045 | 0.0018 | 216264 s at | LAMB2 | laminin, beta 2 (laminin S) |
| 11 | −1.2656 | 0.0314 | 0.0046 | 0.0019 | 221562 s at | SIRT3 | sirtuin (silent mating type information regulation |
| | | | | | | | 2 homolog) 3 (S. cerevisiae) |
| 12 | −1.2628 | 0.0322 | 0.0049 | 0.0022 | 216520 s at | TPT1 | tumor protein, translationally-controlled 1 |
| 13 | −1.2568 | 0.0351 | 0.0056 | 0.0023 | 220141 at | FLJ23554 | hypothetical protein FLJ23554 |
| 14 | −1.2557 | 0.0356 | 0.0059 | 0.0023 | 218483 s at | FLJ21827 | hypothetical protein FLJ21827 |
| 15 | −1.2548 | 0.0364 | 0.0060 | 0.0023 | 221771 s at | HSMPP8 | M-phase phosphoprotein, mpp8 |
| 16 | −1.2399 | 0.0450 | 0.0084 | 0.0029 | 220917 s at | WDR19 | WD repeat domain 19 |
| 17 | −1.2259 | 0.0574 | 0.0117 | 0.0044 | 212695 at | CRY2 | cryptochrome 2 (photolyase-like) |
| 18 | −1.2233 | 0.0593 | 0.0124 | 0.0047 | 213340 s at | KIAA0495 | KIAA0495 |
| 19 | −1.2156 | 0.0652 | 0.0140 | 0.0055 | 213444 at | KIAA0543 | KIAA0543 protein |
| 20 | −1.2144 | 0.0662 | 0.0141 | 0.0057 | 220173 at | C14orf45 | chromosome 14 open reading frame 45 |
| 21 | −1.2139 | 0.0666 | 0.0144 | 0.0059 | 201384 s at | M17S2 | membrane component, chromosome 17, surface |
| | | | | | | | marker 2 (ovarian carcinoma antigen CA125) |
| 22 | −1.2098 | 0.0711 | 0.0152 | 0.0062 | 203156 at | AKAP11 | A kinase (PRKA) anchor protein 11 |
| 23 | −1.2064 | 0.0740 | 0.0163 | 0.0065 | 209407 s at | DEAF1 | deformed epidermal autoregulatory factor 1 |
| | | | | | | | (Drosophila) |
| 24 | −1.2017 | 0.0789 | 0.0179 | 0.0067 | 219469 at | DNCH2 | dynein, cytoplasmic, heavy polypeptide 2 |
| 25 | −1.2003 | 0.0802 | 0.0183 | 0.0069 | 203984 s at | CASP9 | caspase 9, apoptosis-related cysteine protease |
| 26 | −1.1973 | 0.0837 | 0.0190 | 0.0071 | 217844 at | CTDSP1 | CTD (carboxy-terminal domain, RNA polymerase |
| | | | | | | | II, polypeptide A) small phosphatase 1 |
| 27 | −1.1906 | 0.0914 | 0.0212 | 0.0084 | 213397 x at | RNASE4 | ribonuclease, RNase A family, 4 |
| 28 | −1.1896 | 0.0919 | 0.0214 | 0.0088 | 206197 at | NME5 | non-metastatic cells 5, protein expressed in |
| | | | | | | | (nucleoside-diphosphate kinase) |
| 29 | −1.1878 | 0.0941 | 0.0221 | 0.0093 | 219922 s at | LTBP3 | latent transforming growth factor beta binding |
| | | | | | | | protein 3 |
| 30 | −1.1829 | 0.1003 | 0.0247 | 0.0102 | 201383 s at | M17S2 | membrane component, chromosome 17, surface |
| | | | | | | | marker 2 (ovarian carcinoma antigen CA125) |
| 31 | −1.1827 | 0.1007 | 0.0249 | 0.0102 | 206081 at | SLC24A1 | solute carrier family 24 |
| | | | | | | | (sodium/potassium/calcium exchanger), member 1 |
| 32 | −1.1709 | 0.1153 | 0.0296 | 0.0129 | 213266 at | 76P | Gamma tubulin ring complex protein (76p gene) |
| 33 | −1.1707 | 0.1156 | 0.0297 | 0.0129 | 209189 at | FOS | v-fos FBJ murine osteosarcoma viral oncogene |
| | | | | | | | homolog |
| 34 | −1.1679 | 0.1201 | 0.0307 | 0.0142 | 214829 at | AASS | aminoadipate-semialdehyde synthase |
| 35 | −1.1633 | 0.1268 | 0.0337 | 0.0153 | 221123 x at | ZNF395 | zinc finger protein 395 |
| 36 | −1.1625 | 0.1279 | 0.0344 | 0.0155 | 200810 s at | CIRBP | cold inducible RNA binding protein |
| 37 | −1.1612 | 0.1298 | 0.0354 | 0.0157 | 210365 at | RUNX1 | runt-related transcription factor 1 (acute myeloid |
| | | | | | | | leukemia 1; aml1 oncogene) |
| 38 | −1.1602 | 0.1314 | 0.0358 | 0.0158 | 212842 x at | RANBP2L1 | RAN binding protein 2-like 1 |
| 39 | −1.1595 | 0.1324 | 0.0362 | 0.0158 | 213364 s at | SNX1 | Sorting nexin 1 |
| 40 | −1.1586 | 0.1339 | 0.0369 | 0.0161 | 220911 s at | KIAA1305 | KIAA1305 |
| 41 | −1.1539 | 0.1444 | 0.0398 | 0.0175 | 201335 s at | ARHGEF12 | Rho guanine nucleotide exchange factor (GEF) 12 |
| 42 | −1.1499 | 0.1519 | 0.0421 | 0.0190 | 221276 s at | SYNCOILIN | intermediate filament protein syncoilin |
| 43 | −1.1453 | 0.1600 | 0.0451 | 0.0209 | 221824 s at | MIR | c-mir, cellular modulator of immune recognition |
| 44 | −1.1437 | 0.1625 | 0.0461 | 0.0217 | 211943 x at | TPT1 | tumor protein, translationally-controlled 1 |
| 45 | −1.1408 | 0.1681 | 0.0481 | 0.0223 | 218552 at | FLJ10948 | hypothetical protein FLJ10948 |
| 46 | −1.1382 | 0.1743 | 0.0510 | 0.0232 | 220326 s at | FLJ10357 | hypothetical protein FLJ10357 |
| 47 | −1.1254 | 0.2014 | 0.0638 | 0.0296 | 212869 x at | TPT1 | tumor protein, translationally-controlled 1 |
| 48 | −1.1184 | 0.2172 | 0.0722 | 0.0342 | 218648 at | TORC3 | transducer of regulated cAMP response element- |
| | | | | | | | binding protein (CREB) 3 |
| 49 | −1.1171 | 0.2215 | 0.0735 | 0.0348 | 212549 at | STAT5B | signal transducer and activator of transcription |
| | | | | | | | 5B |
| 50 | −1.1165 | 0.2233 | 0.0743 | 0.0356 | 219951 s at | C20orf12 | chromosome 20 open reading frame 12 |
| 51 | −1.1147 | 0.2277 | 0.0763 | 0.0364 | 212678 at | NF1 | Neurofibromin 1 (neurofibromatosis, von Reck |
| | | | | | | | linghausen disease, Watson disease) |
| 52 | −1.1113 | 0.2369 | 0.0807 | 0.0386 | 210852 s at | AASS | aminoadipate-semialdehyde synthase |
| 53 | −1.1112 | 0.2370 | 0.0807 | 0.0387 | 202962 at | KIF13B | kinesin family member 13B |
| 54 | −1.1102 | 0.2404 | 0.0813 | 0.0394 | 214724 at | DIXDC1 | DIX domain containing 1 |
| 55 | −1.1089 | 0.2450 | 0.0823 | 0.0408 | 206542 s at | SMARCA2 | SWI/SNF related, matrix associated, actin dependent |
| | | | | | | | regulator of chromatin, subfamily a, member 2 |
| 56 | −1.1085 | 0.2464 | 0.0827 | 0.0411 | 207757 at | FLJ21628 | hypothetical protein FLJ21628 |
| 57 | −1.1085 | 0.2464 | 0.0827 | 0.0411 | 218466 at | TBC1D17 | TBC1 domain family, member 17 |

TABLE 4
Univariate and Multivariate analysis of breast cancer prognostic markers (N = 417*)

| | Univariate Analysis | | Multivariate Analysis | |
|---|---|---|---|---|
| | Hazard ratio | | Hazard ratio | |
| | (95% CI) | p¶ | (95% CI) | p¶ |
| Age (years) | | | | |
| ≦50 vs >50 | 1.055 (0.556-2.004) | 0.869 | 0.906 (0.416-1.975) | 0.8040 |
| Size | | | | |
| >2 cm vs ≦2 cm | 2.694 (1.618-4.485) | 0.0001 | 2.153 (1.235-3.755) | 0.0068 |
| Histological grade | | | | |
| 1 vs 2 vs 3 | 2.102 (1.461-3.024) | 0.00006 | 1.446 (0.963-2.171) | 0.0754 |
| Estrogen Receptor | | | | |
| Rich vs Poor | 0.937 (0.671-1.307) | 0.937 | 1.212 (0.667-2.202) | 0.5275 |
| Progesterone Receptor | | | | |
| Rich vs Poor | 0.536 (0.381-0.754) | 0.00034 | 0.755 (0.430-1.328) | 0.3300 |
| Genomic Grade | | | | |
| High vs Low | 2.610 (1.833-3.717) | 0.0000001 | 2.302 (1.241-4.271) | 0.0081 |

*Only patients with complete information in all variables were included in the multivariate analysis (N = 208)
¶Based on Cox regression, stratified according to the datasets

TABLE 5
Univariate and Multivariate analysis of breast cancer prognostic markers (N = 249*)

| | Univariate Analysis | | Multivariate Analysis | |
|---|---|---|---|---|
| | Hazard ratio | | Hazard ratio | |
| | (95% CI) | p¶ | (95% CI) | p¶ |
| Age (years) | | | | |
| ≦50 vs >50 | 0.926 (0.328-2.612) | 0.8840 | 0.807 (0.223-2.916) | 0.7440 |
| Size | | | | |
| >2 cm vs ≦2 cm | 2.002 (1.157-3.463) | 0.0130 | 1.712 (0.897-3.268) | 0.1030 |
| Histological grade | | | | |
| 1 vs 2 vs 3 | 1.728 (1.128-2.647) | 0.0120 | 1.071 (0.624-1.839) | 0.8040 |
| Nodal status | | | | |
| Positive vs Negative | 1.444 (0.836-2.493) | 0.1870 | 1.053 (0.554-2.001) | 0.8760 |
| Estrogen Receptor | | | | |
| Rich vs Poor | 0.839 (0.512-1.376) | 0.4860 | 0.982 (0.547-1.764) | 0.9530 |
| Progesterone Receptor | | | | |
| Rich vs Poor | 0.485 (0.291-0.806) | 0.0050 | 0.751 (0.409-1.381) | 0.3570 |
| Genomic Grade | | | | |
| High vs Low | 3.119 (1.861-5.228) | <0.000001 | 2.147 (1.042-4.422) | 0.0380 |

*Only patients with complete information in all variables were included in the multivariate analysis
¶Based on Cox regression, stratified according to the datasets

### REFERENCES

1. Elston C W, et al. Histopathology 1991; 19(5):403-10.
2. Elston C W, et al., Ellis I O. Histopathology 1991; 19; 403-410. Histopathology 2002; 41(3A):151.
3. Galea M H, et al. 1992; 22(3):207-19.
4. Paik S, et al. N Engl J Med 2004; 351(27):2817-26.
5. Robbins P, et al. Hum Pathol 1995; 26(8):873-9.
6. Hopton D S, et al. Eur J Surg Oncol 1989; 15(1):21-3.
7. Theissig F, et al. Pathol Res Pract 1990; 186(6):732-6.
8. Fitzgibbons P L, et al. Arch Pathol Lab Med 2000; 124(7):966-78.
9. Singletary S E, et al. J Clin Oncol 2002; 20(17):3628-36.
10. Perou C M, et al. Nature 2000; 406:747-52.
11. Sorlie T, et al. Proc Natl Acad Science 2001; 98(19):10869-74.
12. Sorlie T, et al. Proc Natl Acad Science 2003; 100(14):8418-23.
13. Sotiriou C, et al. Proc Natl Acad Sci USA 2003; 100(18):10393-8.
14. van de Vijver M J, et al. N Engl J Med 2002; 347(25):1999-2009.
15. Irizarry R A, et al. Biostatistics 2003; 4(2):249-64.
16. Hedges L, Olsen I. Statistical methods for meta-analysis: Academic Press, London; 1985.
17. Korn E L, et al. J Statist Plann Inference 2004; 124:379-398.
18. Praz V, et al. Nucleic Acids Res 2004; 32 (Database issue):D542-7.
19. Ma X J, et al. Proc Natl Acad Sci USA 2003; 100(10):5974-9.
20. van 't Veer L J, et al. Nature 2002; 415(6871):530-6.
21. Wang Y, et al. Lancet 2005; 365:671-79.
22. Ein-Dor L, et al. Bioinformatics 2004; 21(2):171-8.
23. Michiels S, et al. Lancet 2005; 365(9458):488-92.
24. Jenssen T K, et al. Lancet 2005; 365(9460):634-5.
25. Sorlie T, et al. Proc Natl Acad Science. 2003; 100:8418-23
26. Dai H, et al. Cancer Res. 2005; 65:4059-66
27. Sorlie T. Eur J. Cancer. 2004; 40:2667-75
28. Eisen M B, et al. Proc Natl Acad Sci USA 1998; 95:14863-8
29. Therneau T M. Grambasch P M. Modeling Survival Data: Extending the Cox Model. In; 2000.
30. Loi S, et al. (BC). Proc Am Soc Clin Oncol. 2005; 23:6s
31. Clarke R, et al. Oncogene. 2003; 22:7316-39.
32. Wilcken N R, et al. Clin Cancer Res. 1997; 3:849-54.
33. Baum M, et al. Cancer. 2003; 98:1802-10.
34. Boccardo F, Franchi R. American Society of Clinical Oncology. Orlando, Fla. abstract (526); 2005.
35. Goss P E, et al. proc Am Soc Clin Oncol. 2004; 22:88s.
36. Coombes R C, et al. N Engl J. Med. 2004; 350:1081-92.
37. Jakesz R, et al. J. Lancet. 2005; 366:455-62.
38. Effects of chemotherapy and hormonal therapy for early breast cancer on recurrence and 15-year survival: an overview of the randomised trials. Lancet. 2005; 365:1687-717.
39. Winer E P, et al. J Clin Oncol. 2005; 23:619-29.

1. A diagnostic kit or device comprising nucleotide sequences for measuring gene expression of at least 1 gene selected from the genes of table 3 designated as “up regulated genes in grade 1 tumors” the said 1 gene being 13CDNA73.

2. The diagnostic kit or device of claim 1, which further comprises nucleotide sequences for measuring gene expression of at least another gene selected from the genes in table 3 designated as “up regulated genes in grade 1 tumors”.

3. The diagnostic kit or device of claim 1, which further comprises nucleotide sequences for measuring gene expression of at least 4 genes selected from the genes in table 3 designated as “up regulated genes in grade 3 tumors”.

4. The diagnostic kit or device according to claim 1, further comprising means for real time PCR analysis.

5. The diagnostic kit or device according to claim 2, further comprising means for real time PCR analysis.

6. The diagnostic kit or device according to claim 3, further comprising means for real time PCR analysis.

7. The diagnostic kit or device according to claim 4, which further comprises means for real time PCR analysis of reference genes.

8. The diagnostic kit or device according to claim 7, wherein the reference genes are selected from the group consisting of TFRC, GUS, RPLPO and TBP.

9. The kit or device according to claim 1 which is a computerized system comprising:
a) a bio assay module configured for detecting gene expression for a tumor sample based on the gene set according to claim 1 and,
b) a processor module configured to calculate gene-expression grade index (GGI) or relapse score (RS) based on the gene expression and to generate a risk assessment for the tumor sample.

10. A method for the prognosis or diagnosis of cancer in a tumor sample which comprises the step of measuring gene expression of the gene 13CDNA73 in the said tumor sample and correlating the expression of the said gene 13CDNA73 with cancer prognosis or diagnosis.

11. The method of claim 10, which comprises the step of further measuring gene expression of another gene selected from the genes in table 3 designated as “up regulated genes in grade 1 tumors” and correlating the expression of the genes with cancer prognosis or diagnosis.

12. The method of claim 10, which comprises the step of further measuring gene expression of at least 4 genes selected from the genes in table 3 designated as “up regulated genes in grade 3 tumors” and correlating the expression of the genes with cancer prognosis or diagnosis.

13. A method comprising the step of:
a) measuring gene expression in a tumor sample,
b) calculating gene-expression grade index (GGI) of the tumor sample by using the formula:
$${\sum }_{j ∈ {G}_{3}} {x}_{j} - {\sum }_{j ∈ {G}_{1}} {x}_{j}$$
wherein:
x is the gene expression level of mRNA,
G1 and G₃ are sets of genes up-regulated in HG1 and HG3, respectively, and
j refers to a probe or probe set, wherein the gene set comprises the gene 13CDNA73.

14. The method of claim 13, wherein the gene set further comprises another gene selected from the genes in table 3 designated as “up regulated genes in grade 1 tumors”.

15. The method of claim 13, wherein the gene set further comprises at least 4 gene selected from the genes in table 3 designated as “up regulated genes in grade 3 tumors”.

16. The method according to claim 13, wherein the tumor sample is a breast tumor histological grade 2.

17. The method according to claim 13, further comprising the step of designating the breast tumor sample as low risk (GG1) or high risk (GG3) based on the GGI index obtained.

18. The method according to claim 13, further comprising the step of providing a breast cancer treatment regimen for a patient consistent with the low risk or high risk designation of the breast tumor sample.

19. The method according to claim 13, which further comprises a step of designating a breast tumor sample as different subtypes within ER-positive tumors.

20. The method according to claim 13, which further comprises a step of designating a tumor sample as a subtype to be submitted to a different treatment than the other subtype.

21. The method according to claim 13, which is combined to an estrogen receptor and/or progesterone receptor gene expression detection.


SEQUENCE LISTING (24 sequences)

SEQ ID NO: 1 - DNA, 21 bp, Artificial sequence
  Primer MYBL2
agcaagtgcaaggtcaaatgg

SEQ ID NO: 2 - DNA, 20 bp, Artificial sequence
  Primer MYBL2
ctgtccaaactgcctcacca

SEQ ID NO: 3 - DNA, 19 bp, Artificial sequence
  Primer CCNA2
gaagacgagacgggttgca

SEQ ID NO: 4 - DNA, 20 bp, Artificial sequence
  Primer CCNA2
ccaaggaggaacggtgacat

SEQ ID NO: 5 - DNA, 22 bp, Artificial sequence
  Primer STK6
tcttccaggaggaccactctct

SEQ ID NO: 6 - DNA, 21 bp, Artificial sequence
  Primer STK6
tgcatccgaccttcaatcatt

SEQ ID NO: 7 - DNA, 26 bp, Artificial sequence
  Primer KPNA2
taaggcagattttaagacacaaaagg

SEQ ID NO: 8 - DNA, 25 bp, Artificial sequence
  Primer KPNA2
gttcaactgttccaccactggtata

SEQ ID NO: 9 - DNA, 17 bp, Artificial sequence
  Primer CCNB1
catggcgctccgagtca

SEQ ID NO: 10 - DNA, 18 bp, Artificial sequence
  Primer CCNB1
gcgcctgccatgttgatc

SEQ ID NO: 11 - DNA, 16 bp, Artificial sequence
  Primer CDC2
gccgccgcggaataat

SEQ ID NO: 12 - DNA, 26 bp, Artificial sequence
  Primer CDC2
ccttctccaattttctctattttggt

SEQ ID NO: 13 - DNA, 20 bp, Artificial sequence
  Primer CDC20
cttccctgccagaccgtatc

SEQ ID NO: 14 - DNA, 24 bp, Artificial sequence
  Primer CDC20
ccaatccacaaggttcaggtaata

SEQ ID NO: 15 - DNA, 20 bp, Artificial sequence
  Primer MCM2
ggccacaacgtcttcaagga

SEQ ID NO: 16 - DNA, 23 bp, Artificial sequence
  Primer MCM2
atagttcaccaccaggctctcac

SEQ ID NO: 17 - DNA, 20 bp, Artificial sequence
  Primer GUSB
gagtggtgctgaggattggc

SEQ ID NO: 18 - DNA, 20 bp, Artificial sequence
  Primer GUSB
tctagcgtgtcgaccccatt

SEQ ID NO: 19 - DNA, 18 bp, Artificial sequence
  Primer TBP
gcccgaaacgccgaatat

SEQ ID NO: 20 - DNA, 23 bp, Artificial sequence
  Primer TBP
tcgtggctctcttatcctcatga

SEQ ID NO: 21 - DNA, 21 bp, Artificial sequence
  Primer RPLP0
accaaggaggacctcactgag

SEQ ID NO: 22 - DNA, 17 bp, Artificial sequence
  Primer RPLP0
accagcacgggcagcag

SEQ ID NO: 23 - DNA, 20 bp, Artificial sequence
  Primer TFRC
ggagccaggagaggacttcc

SEQ ID NO: 24 - DNA, 24 bp, Artificial sequence
  Primer TFRC
ttctccgacaactttctcttcagg